(19) United States
(12) Patent Application Publication
(10) Pub. No.: US 2026/0294943 A1
(43) Pub. Date: Oct. 1, 2026

(54) LACTO-N-FUCOPENTAOSE AND B. INFANTIS TO PROTECT FROM ENTERIC PATHOGENS

(71) Applicant: SOCIÉTÉ DES PRODUITS NESTLÉ S.A., Vevey (CH)

(72) Inventors: Norbert SPRENGER, Savigny (CH); Kieran JAMES, Bern (CH); Florac DE BRUYN, Lausanne (CH); Hanne Lore Paula TYTGAT, Zurich (CH)

(21) Appl. No.: 19/477,324

(22) PCT Filed: Apr. 26, 2024
(86) PCT No.: PCT/EP2024/061692

§ 371 Date: Oct. 21, 2025

(30) Foreign Application Priority Data
Apr. 28, 2023 (EP) 23170846.2

(51) Int. Cl. A61K 31/702; A61K 35/745; A61K 47/42; A61K 47/44; A61P 31/14

(52) U.S. Cl. CPC A61K 31/702; A61K 35/745; A61K 47/42; A61K 47/44; A61P 31/14

(57) ABSTRACT

The present invention concerns a combination and a nutritional composition comprising the fucosylated human milk oligosaccharide lacto-N-fucopentaose-I (LNFP-I) as the most abundant human milk oligosaccharide in the combination or in the nutritional composition and the Bifidobacterium longum subspecies infantis (B. infantis). The combination and the nutritional composition are useful for treating, preventing and/or inhibiting the growth of enteric pathogens particularly in infants.

### FIELD OF THE INVENTION

The present invention relates to combinations and nutritional compositions especiallyfor infants, young children or children and their health effects. Such combinations and compositions comprise specific oligosaccharide(s) and Bifidobacterium longum and are efficient to protect from enteric pathogens. In particular, it relates to infant formula comprising at least one Lacto-N-fucopentaose as human milk oligosaccharide (HMO) and a Bifidobacterium longum subspecies infantis (B. infantis) that provides protection from different enteric pathogens, namely Salmonella and enteropathogenic E. coli (EPEC) by inhibiting their growth.

### BACKGROUND OF THE INVENTION

Mother's milk is recommended for all infants. However, in some cases breast feeding is inadequate or unsuccessful for medical reasons or the mother chooses not to breast feed. Nutritional compositions such as infant formulae have been developed for these situations.

Nutritional compositions for infants and young children are often sold as powders to be reconstituted with water or in some instances as ready to drink or concentrated liquid compositions. Those compositions are intended to cover most or all the nutritional needs of the infants or young children. It is known however, that human breast milk represents the ultimate gold standard in terms of infants' nutrition. Infant formula manufacturers have therefore made many attempts to induce nutritional health effects close to or similar to the benefits of human breast milk. However, many studies have shown that infant formula do not induce the identical effects on the body compared to human breast milk. For example, infants fed infant formula and infants fed human-breast milk (HBM) can exhibit a different intestinal (gut) microbiota.

Specific Bifidobacterium species like Bifidobacterium longum subsp. Infantis (B. infantis) are amongst the first to colonize the infant gut, partly due to their capacity to metabolize complex human milk oligosaccharides (HMO) and are proposed to play a key role in the development of the infant gut. Strains belonging to B. infantis are particularly well-adapted to metabolize the indigestible human milk oligosaccharides (HMOs), which explains at least in part their abundance in the gastrointestinal tract of breast-fed infants. They are also proposed to play a key role in the maturation of the immune system and the improvement of the gut barrier function in early infancy. It was hypothesized that restoring a B. infantis-dominant microbiota in early infancy, through the co-administration of HMOs and selected B. infantis strains, is beneficial for the infant immune system maturation (Duboux et al. Microorganisms 2022, 10(2), 203). It has been reported that carbohydrate utilization patterns among different strains of B. infantis may vary. Strains belonging to the strain type (ATCC 15697) cluster have been described to consume different types of HMOs simultaneously and equally well with a preference for neutral and sialylated HMOs. Opposed to that, the Bi-26 strain that phylogenetically clusters together with LMG 11588 (ATCC 17930), was shown to be adapted to rapidly internalize and metabolize in particular fucosylated HMOs. Fucosylated HMOs are highly prevalent in human breast milk. 2-fucosyllactose (2′-FL) is the most abundant fucosylated HMO and represents up to 45% of the total HMO content of breast milk, whereas LNFP-I is less represented (Thum et al. Nutrients. 2021 July; 13(7): 2272).

Generally, B. infantis has the ability to efficiently catabolise a wide array of HMO structures, in particular fucosylated ones. Among the major occurring fucosylated HMOs in breastmilk is the pentasaccharide lacto-N-fucopentaose I (LNFP-I) (Garrido et al. Scientific Reports volume 6, Article number: 35045 (2016)).

WO2018/024870 A1 relates to infants or young children nutritional compositions comprising at least one fucosylated oligosaccharide and at least one N-acetylated oligosaccharide for use in preventing and/or treating non-rotavirus diarrhea in infants or young children by acting on the dysbiosis of the microbiota preceding and/or following the non-rotavirus diarrhea due to E. coli (ETEC, EPEC and/or EAEC), Salmonella, Shigella, Aeromonas and/or Campylobacter. The preferred fucosylated oligosaccharide is the widely studied 2′-fucosyllactose (2′FL). Lacto-N-fucopentaose (e.g. lacto-N-fucopentaose I, lacto-N-fucopentaose II, lacto-N-fucopentaose III, lacto-N-fucopentaose V), lacto-N-fucohexaose, lacto-N-difucohexaose I, fucosyllacto-N-hexaose, fucosyllacto-N-neohexaose, difucosyllacto-N-hexaose I, difucosyllacto-N-neohexaose II are cited amongst examples of fucosylated oligosaccharides without any evidence. The addition of probiotics such as Bifidobacterium longum ATCC BAA-999 sold by Morinaga Milk Industry Co. Ltd. Of Japan under the trademark BB536, Bifidobacterium breve sold by Danisco under the trademark Bb-03, Bifidobacterium breve sold by Morinaga under the trademark M-16V, Bifidobacterium infantis sold by Procter & Gamble Co. under the trademark Bifantis is mentioned among other probiotics without any evidence.

WO2021/217803 A1 relates to nutritional compositions comprising an HMO, preferably 2′FL, and a probiotic microorganism comprising Bifidobacterium for reducing intestinal gas production in infants and young children. Lacto-N-fucopentaose (e.g. lacto-N-fucopentaose 1, lacto-N-fucopentaose 11, lacto-N-fucopentaose III and Bifidobacterium infantis are cited amongst examples ofoligosaccharides and probiotic. WO2021/217803 A1 mentions that studies have shown that HMO in general plays a very important role in early growth development in infants, such as 1) regulating the proliferation of intestinal flora, in particular beneficial bacteria (e. g. Bifidobacteria), 2) indirectly inhibiting the growth of pathogenic bacteria by increasing the competitive advantage of non-pathogenic co-organisms, and also directly acting as an anti-adherent antimicrobial agent to reduce microbial infections.

WO2021/061991 A1 relates to the administration of B. longum subsp. Infantis and a concentrated HMO mixture to adult subjects for several benefits such as the reduction in gut domination by pathogenic taxa. Lacto-N-difucohexaose I (LNDFH-I), Lacto-N-difucohexaose-II (LNDFH-II), Lacto-N-fucopentaose-I (LNFP-1), Lacto-N-fucopentaose-II (LNFP-II), Lacto-N-fucopentaose-III (LNFP-III) etc. are mentioned as possible prebiotics.

It is known from WO2021021746A1 that the combination of B. infantis with a milk fat globule membrane complex (MFGM) comprising and LNT and also LNFP provides protection from enteric pathogens.

It can be seen from the prior art that the HMOs lacto-N-fucopentaoses like LNFP-I and Lacto-N-fucohexaoses like LNDFH-I are mentioned but have not extensively been studied so far.

LNFP-I may be isolated by chromatography or filtration technology from a natural source such as animal milks. Suitably, the animal milk as used herein may be cow, sheep, goat, camel or buffalo milk. Preferably, the animal milk is cow's milk. Preferably, the LNFP-I may be produced by biotechnological means using specific fucosyltransferases and/or fucosidases either through the use of enzyme-based fermentation technology (recombinant or natural enzymes) or microbial fermentation technology. In the latter case, microbes may either express their natural enzymes and substrates or may be engineered to produce respective substrates and enzymes. Single microbial cultures and/or mixed cultures may be used. Fucosylated oligosaccharide formation can be initiated by acceptor substrates starting from any degree of polymerization (DP), from DP=1 onwards. Fucosylated oligosaccharides are also available for example from Kyowa, Hakko, Kogyo from Japan, and DSM from the Netherlands or from Elicityl of France. Suitable techniques for producing LNFP-I are known in the art (see, for example, Hu et al., Carbohydr Polym, 2022, 297: 120017 and Derya et al., J Biotechnol., 2020, 318: 31-38). Alternatively, LNFP-I may be produced by chemical synthesis from lactose as initial acceptor substrate building an LNT backbone and free fucose as final donor substrate or by starting from LNT for example, produced by biotechnology or chemical synthesis, and using fucose, but the methods of production are hardly scalable.

There is a need to deliver such health benefits in these infants or young children in a manner that does not induce side effects and/or in a manner that is easy to deliver, and well accepted by the parents or health care practitioners.

### SUMMARY OF THE INVENTION

The inventors have surprisingly found that it was possible to enrich an HMO blend in lacto-N-fucopentaoses as fucosylated human milk oligosaccharides, especially LNFP-I.

The present inventors surprisingly found that a combination of this HMO blend enriched in LNFP-I and B. infantis can advantageously form a combination to be used in therapy because the combination (mixture) showed surprising antibacterial properties.

In other words, the present inventors have found that these specific combinations are particularly effective in increasing the growth and activity of B. infantis in the gastrointestinal tract especially of the infant, young child and/or child, and inhibiting the growth of enteric pathogens. Thus, the invention provides a way to increase the growth and activity of a probiotic (i.e. B. infantis) with known health benefits. The impact of B. infantis in combination with LNFP-I and optionally other HMOs in the gastrointestinal tract was not previously reported.

Accordingly, in a first aspect, the present invention provides a combination comprising at least one fucosylated human milk oligosaccharide and at least one Bifidobacterium longum for use in preventing and/or inhibiting the growth of at least one enteric pathogen in a subject, wherein the at least one fucosylated human milk oligosaccharide comprises lacto-N-fucopentaose I (LNFP-I) as a lacto-N-fucopentaose, wherein LNFP-I in the most abundant HMO in the combination, wherein the at least one Bifidobacterium longum is the subspecies infantis (B. infantis).

In a second aspect, the present invention provides a nutritional composition comprising the combination comprising at least one fucosylated human milk oligosaccharide and at least one Bifidobacterium longum for use in preventing and/or inhibiting the growth of at least one enteric pathogen in a subject, wherein the at least one fucosylated human milk oligosaccharide comprises lacto-N-fucopentaose I (LNFP-I) as a lacto-N-fucopentaose, wherein LNFP-I in the most abundant HMO in the combination, wherein the at least one Bifidobacterium longum is the subspecies infantis (B. infantis).

In some embodiments, the nutritional composition contains other HMOs than the one provided in the combination.

In some embodiments, the nutritional composition does not contain any other HMOs than the one provided in the combination.

In some embodiments, the nutritional composition comprises B. infantis in an amount of 1×10³ to 1.5×10¹² CFU/g of the composition (dry weight).

In some preferred embodiments, the B. infantis strain is NCC 341 (ATCC 15697) and/or LMG 11588 (ATCC 17930).

The fucosylated HMO consumption patterns displayed by B. infantis strains NCC 341 (ATCC 15697) and LMG 11588 (ATCC 17930) show that both strains consume fucosylated HMOs, LMG 11588 to a greater extent (Duboux S, Ngom-Bru C, De Bruyn F, Bogicevic B. Phylogenetic, Functional and Safety Features of 1950s B. infantis Strains. Microorganisms. 2022 Jan. 18; 10(2):203. doi: 10.3390/microorganisms10020203. PMID: 35208658; PMCID: PMC8879182).

In some embodiments, the B. infantis strain has at least 99% Average Nucleotide Identity (ANI) to B. longum subspecies infantis ATCC 15697.

In some embodiments, the B. infantis strain has at least 99% Average Nucleotide Identity (ANI) to B. longum subspecies infantis ATCC 17930.

In some embodiments, the total amount of HMOs in the nutritional composition is ranging from 0.02 to 5 g/100 g dry weight of the nutritional composition.

In some embodiments, the nutritional composition is an infant formula, a starter infant formula, a follow-on or follow-up infant formula, a growing-up milk, a baby food, an infant cereal composition, a (milk) fortifier or a supplement.

In a further aspect, the present invention provides the nutritional composition of the invention for use as a medicament.

In a further aspect, the present invention provides the combination or the nutritional composition of the invention for use in enhancing the immune response to bacterial infection such as EPEC and/or Salmonella, for use in promoting and/or maintaining gut health, or for use in preventing and/or reducing the risk of developing an infection in an infant, young child or a child.

In a further aspect, the present invention provides the use of the combination or of the nutritional composition according to the invention for enhancing the growth of B. infantis in the gastrointestinal tract, or for increasing the levels of at least one or a series of beneficial metabolites.

In a further aspect, the present invention provides a method for promoting immunity in a subject in need thereof, the method comprising administering to the subject a combination or a nutritional composition comprising B. infantis and LNFP-I.

In a further aspect, the present invention provides a combination comprising or consisting of B. infantis and LNFP-I, wherein LNFP-I in the most abundant HMO in the combination, and in further aspects its uses and methods using it.

In a further aspect, the present invention provides a nutritional composition comprising B. infantis and LNFP-I, wherein LNFP-I in the most abundant HMO in the nutritional composition, and in further aspects its uses and methods using it.

In a further aspect, the present invention provides a nutritional composition comprising a combination, the combination comprising or consisting of B. infantis LMG 11588 (ATCC 17930) and LNFP-I, wherein LNFP-I in the most abundant HMO in the nutritional composition, and in further aspects its uses and methods using it.

In a further aspect, the present invention provides a combination comprising or consisting of the strain B. infantis LMG 11588 (ATCC 17930) and LNFP-I, wherein LNFP-I in the most abundant HMO in the combination, and in further aspects its uses and methods using it.

In a further aspect, the present invention provides a nutritional composition comprising the strain B. infantis LMG 11588 (ATCC 17930) and LNFP-I, wherein LNFP-I in the most abundant HMO in the nutritional composition, and in further aspects its uses and methods using it.

In a further aspect, the present invention provides a nutritional composition comprising a combination, the combination comprising or consisting of the strain B. infantis LMG 11588 (ATCC 17930) and LNFP-I, wherein LNFP-I in the most abundant HMO in the nutritional composition, and in further aspects its uses and methods using it.

### BRIEF DESCRIPTION OF THE FIGURES

Additional features and advantages of the present invention are described in, and will be apparent from, the description of the presently preferred embodiments which are set out below with reference to the drawings in which:

FIG. 1 is a HPAEC chromatography representation the HMO blend of example 1, mainly composed of LNFP-I, LNFP-II, LNFP-III, LNDFH-I and LNT.

FIG. 2 reports the results of the growth curve of Salmonella typhymurium in presence of the different spent supernatants as indicated.

FIG. 3 reports the pate enumeration of Salmonella typhymurium after incubation in presence of the different spent supernatants as indicated.

FIG. 4. reports the pate enumeration of Enteropathogenic E. coli (EPEC) after incubation in presence of the different spent supernatants as indicated.

In FIGS. 2-4 “HMO” means the HMO blend of example 1, and “Blnf” means B. infantis.

FIG. 5. Biomass growth with LNFP-I and Bifidobacterium longum subsp. infantis LMG 11588 (NCC3039) compared to other HMOs. LNFP-I presents a unique diauxic shift. Experiments were conducted in triplicate, with the average being depicted.

### DETAILED DESCRIPTION OF THE INVENTION

### Definitions

Within the context of the present invention, the following terms have the following meanings.

The term “infant” means a child underthe age of 12 months. The expression “young child” means a child aged between one and less than three years, also called toddler. The expression “child” means a child between three and nine years of age. Preferably, the expression “child” means a child between three and five years of age.

The expression “nutritional composition” means a composition which nourishes a subject. This nutritional composition is usually to be taken orally or parenterally, and it usually includes a lipid or fat source and a protein source. A carbohydrate source may also be included. In one embodiment, the nutritional composition of the invention is a synthetic nutritional composition.

In a particular embodiment, the combination or composition of the present invention is a “synthetic combination” or “synthetic nutritional composition”. The expression “synthetic combination” or “synthetic nutritional composition” means a mixture obtained by chemical and/or biological means, which can be chemically identical to the mixture naturally occurring in mammalian milks (i.e. the synthetic combination or synthetic composition is not breast milk).

The expression “infant formula” as used herein refers to a foodstuff intended for particular nutritional use by infants during the first months of life and satisfying by itself the nutritional requirements of this category of person (Article 2(c) of the European Commission Directive 91/321/EEC 2006/141/EC of 22 Dec. 2006 on infant formulae and follow-on formulae). It also refers to a nutritional composition intended for infants and as defined in Codex Alimentarius (Codex STAN 72-1981) and Infant Specialities (incl. Food for Special Medical Purpose). The expression “infant formula” encompasses both “starter infant formula” and “follow-up formula” or “follow-on formula”.

A “follow-up formula” or “follow-on formula” is given from the 6th month onwards. It constitutes the principal liquid element in the progressively diversified diet of this category of person.

The expression “baby food” means a foodstuff intended for particular nutritional use by infants or young children during the first years of life.

The expression “infant cereal composition” means a foodstuff intended for particular nutritional use by infants or young children during the first years of life.

The expression “growing-up milk” (or GUM) refers to a milk-based drink generally with added vitamins and minerals, that is intended for young children or children.

The term “fortifier” refers to liquid or solid nutritional compositions suitable for fortifying or mixing with human milk, infant formula, growing-up milk or human breast milk fortified with other nutrients. Accordingly, the fortifier of the present invention can be administered after dissolution in human breast milk, in infant formula, in growing-up milk or in human breast milk fortified with other nutrients or otherwise it can be administered as a stand-alone composition. When administered as a stand-alone composition, the milk fortifier of the present invention can be also identified as being a “supplement”. In one embodiment, the milk fortifier of the present invention is a supplement.

The expression “weaning period” means the period during which the mother's milk is substituted by other food in the diet of an infant or young child.

The expressions “days/weeks/months/years of life” and “days/weeks/months/years after birth” can be used interchangeably.

The “mother's milk” should be understood as the breast milk or the colostrum of the mother.

An “oligosaccharide” is a saccharide polymer containing a small number (typically three to ten) of simple sugars (monosaccharides).

The expression “nutritional composition” means a composition which nourishes a subject. This nutritional composition is usually to be taken orally, and it usually includes a carbohydrate source, a lipid or fat source and a protein source. In one embodiment, the nutritional composition of the invention is a synthetic nutritional composition.

In a particular embodiment the nutritional composition of the present invention is a “synthetic nutritional composition”. The expression “synthetic nutritional composition” means a mixture obtained by chemical and/or biological means (i.e. the synthetic composition is not breast milk).

The expression “infant formula” as used herein refers to a foodstuff intended for particular nutritional use by infants during the first months of life and satisfying by itself the nutritional requirements of this category of person (Article 2(c) of the European Commission Directive 91/321/EEC 2006/141/EC of 22 Dec. 2006 on infant formulae and follow-on formulae). It also refers to a nutritional composition intended for infants and as defined in Codex Alimentarius (Codex STAN 72-1981) and Infant Specialities (incl. Food for Special Medical Purpose). The expression “infant formula” encompasses both “starter infant formula” and “follow-up formula” or “follow-on formula”.

A “follow-up formula” or “follow-on formula” is given from the 6th month onwards. It constitutes the principal liquid element in the progressively diversified diet of this category of person.

The expression “baby food” means a foodstuff intended for particular nutritional use by infants or young children during the first years of life.

The expression “infant cereal composition” means a foodstuff intended for particular nutritional use by infants or young children during the first years of life.

The expression “growing-up milk” (or GUM) refers to a milk-based drink generally with added vitamins and minerals, that is intended for young children or children.

The term “fortifier” refers to liquid or solid nutritional compositions suitable for fortifying or mixing with human milk, infant formula, growing-up milk, or human breast milk fortified with other nutrients. Accordingly, the fortifier of the present invention can be administered after dissolution in human breast milk, in infant formula, in growing-up milk or in human breast milk fortified with other nutrients or otherwise it can be administered as a stand-alone composition. When administered as a stand-alone composition, the milk fortifier of the present invention can be also identified as being a “supplement”. In one embodiment, the milk fortifier of the present invention is a supplement.

The expression “weaning period” means the period during which the mother's milk is substituted by other food in the diet of an infant or young child.

The term “HMO” or “HMOs” refers to human milk oligosaccharide(s). These carbohydrates are highly resistant to enzymatic hydrolysis, indicating that they may display essential functions not directly related to their caloric value. It has especially been illustrated that they play a vital role in the early development of infants and young children, such as the maturation of the immune system. Many different kinds of HMOs are found in the human milk. Each individual oligosaccharide is based on a combination of glucose, galactose, sialic acid (N-acetylneuraminic acid), fucose and/or N-acetylglucosamine with many and varied linkages between them, thus accounting for the enormous number of different oligosaccharides in human milk—over 130 such structures have been identified so far. Almost all of them have a lactose moiety at their reducing end while sialic acid and/or fucose (when present) occupy terminal positions at the non-reducing ends. The HMOs can be acidic (e.g. charged sialic acid containing oligosaccharide) or neutral (e.g. fucosylated oligosaccharide). A “fucosylated oligosaccharide” is an oligosaccharide having a fucose residue. It has a neutral nature. Some examples are 2′-FL (2′-fucosyllactose), 3-FL (3-fucosyllactose), difucosyllactose, lacto-N-fucopentaose (e.g. lacto-N-fucopentaose-I, lacto-N-fucopentaose-II, lacto-N-fucopentaose-III, lacto-N-fucopentaose-V), lacto-N-fucohexaose, lacto-N-difucohexaose-I (LNDFH-I), fucosyllacto-N-hexaose, fucosyllacto-N-neohexaose, difucosyllacto-N-hexaose-I, difucosyllacto-N-neohexaose-II and any combination thereof.

The expressions “fucosylated oligosaccharides comprising an alpha-linked-fucosyl-epitope” and “2-fucosylated oligosaccharides” encompass fucosylated oligosaccharides with a certain homology of form since they contain an alpha-1,2 linked-fucosyl-epitope, therefore a certain homology of function can be expected.

The expression “N-acetylated oligosaccharide(s) encompasses both “N-acetyl-lactosamine” and “oligosaccharide(s) containing N-acetyl-lactosamine”. They are neutral oligosaccharides having an N-acetyl-lactosamine residue. Suitable examples are LNT (lacto-N-tetraose), para-lacto-N-neohexaose (para-LNnH), LNnT (lacto-N-neotetraose) and any combinations thereof. Other examples are lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose.

The nutritional composition of the present invention can be in solid form (e.g. powder) or in liquid form. The amount of the various ingredients (e.g. the oligosaccharides) can be expressed in g/100 g of composition on a dry weight basis when it is in a solid form, e.g. a powder, or as a concentration in g/L of the composition when it refers to a liquid form (this latter also encompasses liquid composition that may be obtained from a powder after reconstitution in a liquid such as milk, water . . . , e.g. a reconstituted infant formula or a follow-on/follow-up formula or a growing-up milk or an infant cereal product or any other formulation designed for infant nutrition).

The term “galacto-oligosaccharides” refers to a type of non-digestible fiber with prebiotic activity. GOS are formed via enzymatic conversion of lactose. GOS generally comprise a chain of galactose units that arise through consecutive transgalactosylation reactions, with a terminal glucose unit, although a terminal galactose unit may be present instead. The degree of polymerization of GOS typically ranges from 2 to 8 monomeric units.

The nutritional composition of the present invention can be in solid form (e.g. powder) or in liquid form. The amount of the various ingredients (e.g. the oligosaccharides) can be expressed in g/100 g of composition on a dry weight basis when it is in a solid form, e.g. a powder, or as a concentration in g/L of the composition when it refers to a liquid form (this latter also encompasses liquid composition that may be obtained from a powder after reconstitution in a liquid such as milk, water . . . , e.g. a reconstituted infant formula or a follow-on/follow-up formula or an infant cereal product or any other formulation designed for infant nutrition). They can also be expressed in g/100 kcal.

The term “prebiotic” means non-digestible carbohydrates that beneficially affect the host by selectively stimulating the growth and/or the activity of healthy bacteria such as bifidobacteria in the colon of humans (Gibson G R, Roberfroid M B. Dietary modulation of the human colonic microbiota: introducing the concept of prebiotics. J Nutr. 1995; 125:1401-12).

The term “probiotic” means microbial cell preparations or components of microbial cells with a beneficial effect on the health or well-being of the host. (Salminen S, Ouwehand A. Benno Y. et al. “Probiotics: how should they be defined” Trends Food Sci. Technol. 1999:10 107-10). The microbial cells are generally bacteria or yeasts.

Bifidobacterium longum subspecies infantis (B. infantis) is a Gram-positive, heterofermentative, anaerobic bacteria with a distinctive bifid (i.e., “Y”) shape of the Bifidobacteria are genus which can be found in the gastrointestinal tract of humans (Nutrients. 2020 June; 12(6): 1581). B. infantis is sold by Procter & Gamble Co. under the trademark Bifantis. B. infantis can be a strain belonging to the type strain (ATCC 15697) or a Bi-26 strain, clustering together with LMG 11588 (ATCC 17930).

Bifidobacterium longum is a bacterium of the Bifidobacterium genus which is present in the human gastrointestinal tract. In 2002, three previously distinct species of Bifidobacterium, B. infantis, B. longum, and B. suis, were unified into a single species named B. longum with the biotypes infantis, longum, and suis, respectively (Sakata, S., et al., 2002. International journal of systematic and evolutionary microbiology, 52(6), pp. 1945-1951).

Any suitable Bifidobacterium longum subsp. infantis strain may be used in the present invention. Such strains will be well-known to the skilled person. Suitable strains include Bifidobacterium longum subsp. infantis LMG 11588 (also known as Bifidobacterium longum subsp. infantis NCC3039 or Bifidobacterium longum subsp. infantis ATCC 17930) and Bifidobacterium longum subsp. infantis ATCC 15697 (also known as Bifidobacterium longum subsp. infantis NCC 3078), Rosell-33 (sold by Lallemand), m-63 (sold by Morinaga).

The Bifidobacterium longum subsp. infantis may be a strain having at least 99% (suitably, at least 99.9%) ANI to Bifidobacterium longum subsp. infantis strain known to the skilled person.

Suitably, the Bifidobacterium longum subsp. infantis has at least 99% (suitably, at 5 least 99.1%, at least 99.2%, at least 99.3%, at least 99.4%, at least 99.5%, at least 99.6%, at least 99.7%, at least 99.8%, at least 99.9%) ANI to Bifidobacterium longum subsp. infantis LMG 11588 (also known as Bifidobacterium longum subsp. infantis NCC3039 or Bifidobacterium longum subsp. infantis ATCC 17930). Preferably, the Bifidobacterium longum subsp. infantis has at least 99.9% ANI to Bifidobacterium longum subsp. infantis LMG 11588.

Bifidobacterium longum subsp. infantis LMG 11588 is openly available and can be obtained bythe Belgian Coordinated Collections of Microorganisms (BCCM) underthe LMG accession number LMG 11588.

Bifidobacterium longum subsp. infantis ATCC 15697 is openly available and can be obtained by ATCC as ATCC® 15697™

The term “cfu” should be understood as colony-forming unit.

“Treating” means to address a medical condition or disease with the objective of improving or stabilising an outcome in the person being treated or addressing an underlying nutritional need. Treating, therefore, includes the dietary or nutritional management of the medical condition or disease by addressing nutritional needs of the person being treated. Treating includes the elimination, reduction or amelioration of symptoms associated with the medical condition or disease.

“Preventing” means to diminish the risk of onset or recurrence of a medical condition or disease. Both primary and secondary prevention are thus contemplated.

“Primary prevention” means preventing a medical condition or disease before it occurs, and “secondary prevention” means preventing additional attacks of a medical condition or disease after the first attack has occurred. Preventing, therefore, includes the dietary or nutritional prophylaxis of the medical condition or disease by addressing nutritional needs of the person being treated. Preventing includes eliminating or minimising the risk of developing a medical condition or disease and reducing the risk of developing symptoms associated with the medical condition or disease.

The terms “pathogen” and “pathogenic” in reference to a microorganism includes any such microorganism that is capable of causing or affecting a disease, disorder or condition of a host containing the microorganism. Examples of pathogen are enteric pathogens such as Salmonella and enteropathogenic E. coli (EPEC).

The term “effective amount” preferably means an amount of the combination or composition that provides the active agents in a sufficient amount to render a desired treatment or prevention outcome in a subject. An effective amount can be administered in one or more doses to the subject to achieve the desired treatment or prevention outcome.

The term “SCFA” means short chain fatty acid(s).

The expression “increasing SCFA production” means that the amount of systemic and/or colonic SCFA, is higher in an individual fed with the nutritional composition according to the present invention in comparison with a standard. The SCFA production may be measured by techniques known by the skilled person such as by Gas-Liquid Chromatography.

The expression “LNFP-I in the most abundant HMO” in the combination or in the nutritional composition means that LNFP-I is present in an amount superior to the amount of any other HMO present in said combination or nutritional composition.

The “Average Nucleotide Identity (ANI)” is a measure of nucleotide-level genomic similarity between the coding regions of two genomes. Average Nucleotide Identity can be assessed as describe here: Yoon S H, Ha S M, Lim J, Kwon S, Chun J. A large-scale evaluation of algorithms to calculate average nucleotide identity. Antonie Van Leeuwenhoek. 2017 October; 110(10):1281-1286. In the present embodiment the strain Bifidobacterium longum subsp. infantis LMG 11588 (also known as ATCC 17930) represents the reference genome to which a microbial genome is compared. An example of a microorganism genome that has at least 99.9% ANI with B. longum subsp. infantis LMG 11588 can be found in PATRIC (https://www.patricbrc.org), genome ID 1678.111. In one embodiment of the present invention, the Bifidobacterium longum subsp. infantis strain does not harbour potentially transferable antibiotic resistances.

The ANI of the shared genes between two strains is known to be a robust means to compare genetic relatedness among strains. Strains with ANI values of at least about 96% can be considered to belong to the same species (Konstantinidis and Tiedje, 2005, Proc Natl Acad Sci USA, 102(7):2567-72; and Goris et al., 2007, Int Syst Evol Microbiol. 57(Pt 1):81-91), while ANI values of at least about 99% indicate that the bacterial genomes belong to the same strain. The ANI between two bacterial genomes is calculated from pair-wise comparisons of all sequences shared between any two strains and can be determined, for example, using any of a number of publicly available ANI tools, including but not limited to OrthoANI with usearch (Yoon et al., 2017, Antonie van Leeuwenhoek 110:1281-1286); ANI Calculator, JSpecies (Richter and Rossello-Mora, 2009, Proc Natl Acad Sci USA 106:19126-19131); and JSpeciesWS (Richter et al., 2016, Bioinformatics 32:929-931). Other methods for determining the ANI of two genomes are known in the art (Konstantinidis, K. T. and Tiedje, 2005, J. M., Proc. Natl. Acad. Sci. U.S.A., 102: 2567-2572; and Varghese et al., 2015, Nucleic Acids Research, 43(14):6761-6771).

All percentages are by weight unless otherwise stated.

In addition, in the context of the invention, the terms “comprising” or “comprises” do not exclude other possible elements. The composition of the present invention, including the many embodiments described herein, can comprise, consist of, or consist essentially of the essential elements and limitations of the invention described herein, as well as any additional or optional ingredients, components, or limitations described herein or otherwise depending on the needs.

According to the invention, any embodiments, including any preferred, more preferred, most preferred, even more preferred or exemplifying (e.g. with expressions like such as or for example) embodiments, can be combined with each other. For example, a preferred embodiment of a feature can be combined with a more preferred embodiment of another feature.

It should be understood that various changes and modifications to the presently preferred embodiments described herein will be apparent to those skilled in the art. Such changes and modifications can be made without departing from the spirit and scope of the present invention and without diminishing its attendant advantages. It is therefore intended that such changes and modifications be covered by the appended claims.

A first object of the present invention is therefore a combination comprising at least one fucosylated human milk oligosaccharide (HMO) and at least one Bifidobacterium longum for use in treating, preventing and/or inhibiting the growth of at least one enteric pathogen in a subject, wherein the at least one fucosylated human milk oligosaccharide comprises lacto-N-fucopentaose-I (LNFP-1) as a lacto-N-fucopentaose, wherein LNFP-I in the most abundant HMO in the nutritional composition, wherein the least one Bifidobacterium longum is the subspecies infantis (B. infantis).

In some embodiments, the human milk oligosaccharides in the combination have a degree of polymerization (DP) of at least 4, i.e. are at least tetrasaccharides. In some embodiments, each human milk oligosaccharide in the nutritional composition having a degree of polymerization (DP) of less than 4 represent less than 2% by weight of the total amount of human milk oligosaccharides in the combination.

In some embodiments in the combination, lacto-N-fucopentaose I (LNFP-1) represents at least 5% by weight, preferably at least 20% by weight of the total amount of human milk oligosaccharides in the combination on a dry weight basis, preferably from 20% to 100%, more preferably from 25% to 95%, such as from 50% to 95%.

In some embodiments, the combination is further comprising at least one further lacto-N-fucopentaose, preferably lacto-N-fucopentaose 11 and/or lacto-N-fucopentaose III as fucosylated human milk oligosaccharide, preferably both.

In some embodiments, the combination is further comprising as fucosylated human milk oligosaccharide at least one fucohexaose.

In some embodiments the total amount of lacto-N-fucopentaose(s) represents at least 5% by weight, preferably at least 20% by weight of the total amount of human milk oligosaccharides in the combination on a dry weight basis, preferably from 15% to 100%, more preferably from 30% to 95%.

In some embodiments the at least one fucosylated human milk oligosaccharide comprises at least 5% by weight, preferably at least 10% by weight of the total amount of human milk oligosaccharides in the combination on a dry weight basis, of at least one fucohexaose, preferably from 5% to 90%, more preferably from 10% to 80%, such as from 10% to 20%.

In some embodiments of the combination the at least one fucosylated human milk oligosaccharide comprises at least Lacto-N-fucohexaose-I (LNDFH-1) as a fucohexaose.

In some embodiments the at least one enteric pathogen is Salmonella typhymurium.

In some embodiments the at least one enteric pathogen is enteropathogenic E. coli (EPEC).

In some embodiments the at least one Bifidobacterium longum subspecies infantis (B. infantis) is strain LMG 11588, ATCC 15697 (NCC 341) or a mixture thereof.

In some embodiments the combination is further comprising at least one N-acetylated human milk oligosaccharide, preferably Lacto-N-tetraose (LNT), preferably from 0.1% to 30%, more preferably from 10% to 20% by weight of the total amount of human milk oligosaccharides in the combination on a dry weight basis.

In some embodiments the combination or nutritional composition is further comprising at least one N-acetylated human milk oligosaccharide, preferably Lacto-N-tetraose (LNT), Lacto-N-neotetraose (LNnT) or a mixture thereof, preferably from 1% to 50%, more preferably from 5% to 80%, even more preferably from 10% to 60% by weight of the total amount of human milk oligosaccharides in the combination on a dry weight basis. In some embodiments the nutritional composition is an infant formula, a starter infant formula, a follow-on or follow-up infant formula, a growing-up milk, a baby food, an infant cereal composition, a (milk) fortifier or a supplement.

In some embodiments the combination or nutritional composition does not comprise any N-acetylated human milk oligosaccharide such as Lacto-N-tetraose (LNT), Lacto-N-neotetraose (LNnT) or a mixture thereof.

In some embodiments the combination or nutritional composition does not comprise Lacto-N-tetraose (LNT).

In some embodiments the combination or nutritional composition does not comprise Lacto-N-neotetraose (LNnT).

In some embodiments the nutritional composition the subject is a infant, a young child or a child.

In some embodiments, LNFP-I is present in a total amount of from 20 mg/L to 5000 mg/L of the nutritional composition according to the invention after reconstitution in water, or of from 0.02 g/100 g to 4 g/100 g of the nutritional composition or combination according to the invention. Suitably, LNFP-I is present in a total amount of from 50 mg/L to 2500 mg/L, for example from 60 mg/L to 2000 mg/L, for example from 80 mg/L to 1000 mg/L of the nutritional composition or combination according to the invention. Suitably, LNFP-I is present in a total amount of from 0.04 g/100 g to 2 g/100 g, for example from 0.05 g/100 g to 1.6 g/100 g, for example from 0.07 g/100 g to 0.8 g/100 g of the nutritional composition or combination (dry weight).

In some embodiments, LNDFH-I is the next most abundant HMO in the combination after LNFP-I. Hence, in some embodiments LNDFH-I and LNFP-I and the two most abundant HMOs in the combination.

In some embodiments, LNDFH-I is the next most abundant HMO in the combination after LNFP-I and LNFP-III.

In some embodiments, LNDFH-I is the next most abundant HMO in the HMO blend of the combination after LNFP-I, LNFP-II and LNFP-III.

In some embodiments, LNDFH-I is the next most abundant HMO in the HMO blend of the combination after LNFP-I, LNFP-II, LNT and LNFP-III.

In some embodiments, the HMO blend of the combination is comprising LNFP-I in an amount of from 15% to 100% by weight of the total HMOs in the blend, preferably 20% to 95%, more preferably 25% to 90%, for example 25% to 30%, such as 27.5%.

In some embodiments, the HMO blend of the combination is comprising LNFP-II in an amount of from 5% to 50% by weight of the total HMOs in the blend, preferably 10% to 35%, more preferably 10% to 20%, for example 10% to 15%, such as 13%.

In some embodiments, the HMO blend of the combination is comprising LNFP-III in an amount of from 5% to 50% by weight of the total HMOs in the blend, preferably 10% to 35%, more preferably 10% to 20%, for example 12% to 18%, such as 16.3%.

In some embodiments, the HMO blend of the combination is comprising LNDFH-I in an amount of from 5% to 80% by weight of the total HMOs in the blend, preferably 10% to 35%, more preferably 10% to 20%, for example 10% to 15%, such as 13.2%.

In some embodiments, the HMO blend of the combination is comprising LNT in an amount of from 5% to 50% by weight of the total HMOs in the blend, preferably 10% to 35%, more preferably 10% to 20%, for example 12% to 16%, such as 14.1%.

In some embodiments, the HMO blend of the combination is comprising of LNT, LNFP-I, LNFP-II, LNFP-III and LNDFH-I, and any other HMO, if present in the HMO blend, is present in less than 5% by weight of the total HMOs in the blend, preferably 3.5% or less, more preferably 2.0% or less, even more preferably 1.0% or less.

In some embodiments, the HMO blend of the combination are consisting of LNT, LNFP-I, LNFP-II, LNFP-III and LNDFH-I.

In one embodiment, the HMO blend of the combination are comprising of LNFP-I, LNT, LNFP-II, LNFP-III and LNDFH-I.

In some embodiments, the HMO of the combination is consisting of LNFP-I.

In some embodiments, the HMO blend of the combination are consisting of LNFP-I and LNDFH-I.

In some embodiments, the HMO blend of the combination are consisting of LNFP-I, LNFP-II and LNDFH-I.

In some embodiments, the HMO blend of the combination are consisting of LNFP-I, LNFP-III and LNDFH-I.

In one embodiment, the HMO blend of the combination are consisting of LNFP-I, LNFP-II, LNFP-III and LNDFH-I.

In one embodiment, the HMO blend of the combination are consisting of LNFP-I and LNT.

In one embodiment, the HMO blend of the combination are consisting of LNFP-I, LNT and LNDFH-I.

In one embodiment, the HMO blend of the combination are consisting of LNFP-I, LNT, LNFP-II and LNDFH-I.

In one embodiment, the HMO blend of the combination are consisting of LNFP-I, LNT, LNFP-III and LNDFH-I.

In one embodiment, 2′FL and 3FL represent less than 5% by weight of the total amount of human milk oligosaccharides in the combination, in a dry basis preferably 3.5% or less, more preferably 2% or less.

In one embodiment, 2′FL represents less than 3.5% by weight of the total amount of human milk oligosaccharides in the combination, in a dry matter basis preferably 2% or less, most preferably at 1% or less.

In one embodiment, 3FL represents less than 1.5% by weight of the total amount of human milk oligosaccharides in the combination, in a dry basis preferably 0.5% or less, most preferably at most 0.2%.

In one embodiment, 2′FL and 3FL represent less than 5% by weight of the total amount of human milk oligosaccharides in the nutritional composition, in a dry basis preferably 3.5% or less, more preferably 2% or less.

In one embodiment, 2′FL represents less than 3.5% by weight of the total amount of human milk oligosaccharides in the nutritional composition, in a dry basis preferably 2% or less, most preferably at 1% or less.

In one embodiment, 3FL represents less than 1.5% by weight of the total amount of human milk oligosaccharides in the nutritional composition, in a dry basis preferably 0.5% or less, most preferably at most 0.2%.

In some embodiments, the human milk oligosaccharide(s) is present in the nutritional composition according to the present invention in a total amount of 0.5-3 g/L such as 0.8-1.5 g/L of the composition. In some embodiments, the human milk oligosaccharide(s) may be in a total amount of 0.85-1.3 g/L of the composition, such as 0.9-1.25 g/L or 0.9-1.1 g/L or 1-1.25 g/L or 1-1.2 g/L of the composition.

The fucosylated human milk oligosaccharide(s) can be present in the nutritional composition according to the present invention in a total amount of 0.5-3 g/L such as 0.8-1.5 g/L of the composition. In some embodiments, the fucosylated human milk oligosaccharide(s) may be in a total amount of 0.85-1.3 g/L of the composition, such as 0.9-1.25 g/L or 0.9-1.1 g/L or 1-1.25 g/L or 1-1.2 g/L of the composition.

In a further aspect, the invention concerns a nutritional composition comprising the HMO blend as defined in any of the preceding embodiments, and B. infantis.

In a further aspect, the invention concerns a nutritional composition comprising the HMO blend as defined in any of the preceding embodiments, and the strain B. infantis LMG 11588 (ATCC 17930).

In a further aspect, the invention concerns a combination comprising or consisting of the HMO blend as defined in any of the preceding embodiments, and B. infantis.

In a further aspect, the invention concerns a combination comprising or consisting of the HMO blend as defined in any of the preceding embodiments, and the strain B. infantis LMG 11588 (ATCC 17930).

In a further aspect, the invention concerns a nutritional composition comprising a combination, the combination comprising or consisting of the HMO blend as defined in any of the preceding embodiments, and B. infantis.

In a further aspect, the invention concerns a nutritional composition comprising a combination, the combination comprising or consisting of the HMO blend as defined in any of the preceding embodiments, and the strain B. infantis LMG 11588 (ATCC 17930).

In some embodiments the combination can be prepared by dry mixing, wet mixing, or combinations thereof of the HMO(s) comprising or consisting of LNFP-I and B. infantis by standard methods known in the art. Dry powder of B. infantis can be prepared by freeze drying or spray drying. The HMO(s) can be purified/prepared by crystallizations, spray drying, freeze drying, or other drying equipment known in the art.

In some embodiments, the nutritional composition is provided to the infants to protect the gut from opportunistic pathogen invasion (i.e., to provide colonization resistance) at a time where their adaptive immune system is developing.

In some embodiments, the enteric pathogen is a bacterial infection or gut domination comprising an infection or gut domination by one or more species, subspecies, or strains of Aeromonas, Bacillus, Blautia, Bordetella, Borrelia, Brucella, Burkholderia, Campylobacter, Chlamydia, Chlamydophila, Citrobacter, Clostridium, Coiynebacterium, Coxiella, Ehrlichia, Enterobacter, Enterobacteriaceae, Enterococcus, Escherichia, Faecalicatena, Francisella, Haemophilus, Helicobacter, Hungateha, Klebsiella, Lachnospiraceae, Legionella, Leptospira, Listeria, Morganella, Mycobacterium, Mycoplasma, Neisseria, Orientia, Plesiomonas, Proteus, Pseudomonas, Rickettsia, Salmonella, Shigella, Staphylococcus, Streptococcus, Treponema, Vibrio, or Yersinia, optionally one or more of Aeromonas hydrophila, Bacillus cereus, Campylobacter fetus, Campylobacter jejuni, Clostridium botulinum, Clostridium difficile, Clostridium perfringens, enteroaggregative Escherichia coli, enterohemorrhagic Escherichia coli, enteroinvasive Escherichia coli, enteropathogenic E. coli (EPEC), enterotoxigenic Escherichia coli, Escherichia coli O157:H7, Helicobacter pylori, Klebsiellia pneumonia, Lysteria monocytogenes, Salmonella typhymurium, Salmonella paratyphi, Salmonella typhi, Salmonella enterica, Staphylococcus aureus, Vibrio cholerae, Vibrio parahaemolyticus, Vibrio vulnificus, or Yersinia enterocolitica, preferably Salmonella typhymurium and/or EPEC.

In certain embodiments, the enteric pathogen is a bacterial infection or gut dominating bacterium and/or opportunistic pathogen comprising an infection or gut domination caused by drug-resistant bacteria. In some embodiments, the drug-resistant bacteria comprise one or more of antibiotic-resistant bacteria (ARB), Antibiotic-resistant Proteobacteria, Caibapenem-resistant Enterobacteriaceae (CRE), Extended Spectrum Beta-Lactamase producing Enterobacteriaceae (ESBL-E), fluoroquinolone-resistant Enterobacteriaceae, extended spectrum beta-lactam resistant Enterococci (ESBL), vancomycin-resistant Enterococci (VRE), multi-drug resistant E. coli, or multi-drug resistant Klebsiella.

In a further aspect, the present invention provides a non-therapeutic use of a nutritional composition or a combination comprising at least one fucosylated human milk oligosaccharide and at least one Bifidobacterium longum, wherein the at least one fucosylated human milk oligosaccharide comprises lacto-N-fucopentaose I (LNFP-1) as a lacto-N-fucopentaose, wherein LNFP-I in the most abundant HMO in the nutritional composition or in the combination, wherein the least one Bifidobacterium longum is the subspecies infantis (B. infantis), preferably LMG 11588, ATCC 15697 (NCC 341) or a mixture thereof, for treating, preventing and/or inhibiting the growth of at least one enteric pathogen in a subject, wherein the pathogen is preferably enteropathogenic E. coli (EPEC) and/or Salmonella typhymurium, wherein the nutritional composition is preferably an infant formula, a starter infant formula, a follow-on or follow-up infant formula, a growing-up milk, a baby food, an infant cereal composition, a (milk) fortifier or a supplement, wherein the subject is preferably an infant, a young child or a child. All embodiments of the nutritional composition or combination are applicable to the non-therapeutic use.

In a further aspect, the present invention provides a method for promoting immunity in a subject in need thereof, the method comprising administering to the subject a combination or a nutritional composition according to the invention.

In some embodiments, the nutritional composition comprises B. infantis in an amount of 1×10³ to 1.5×10¹² CFU/g of the composition (dry weight).

Various health effects of B. infantis as a probiotic are well documented (references above cited).

The nutritional composition according to the invention may contain from 10³ to 10¹² cfu of B. infantis, more preferably between 10⁷ and 10¹² cfu such as between 10⁸ and 10¹⁰ cfu of B. infantis per g of composition on a dry weight basis. Suitably, B. infantis is administered to the subject in an amount of at least about 10⁶ cfu/day, at least about 10⁷ cfu/day, or at least about 10⁸ cfu/day. Suitably, B. infantis is administered to the subject in an amount of about 10¹² cfu/day or less, about 10¹¹ cfu/day or less, or about 10¹⁰ cfu/day or less.

In one embodiment B. infantis is viable. In another B. infantis is non-replicating or inactivated. B. infantis may be both viable and inactivated in some other embodiments.

The invention will now be described in further details. It is noted that the various aspects, features, examples and embodiments described in the present application may be compatible and/or combined together.

The inventors have surprisingly found that it was possible to enrich an HMO blend in lacto-N-fucopentaoses, especially LNFP-I.

The present inventors surprisingly found that a combination of this HMO blend enriched in LNFP-I and B. infantis form a combination which can advantageously be used in therapy because the combination showed surprising antibacterial properties.

In some embodiments of the combination, the weight ratio of LNFP-I:LNDFH-I is from 90:10 to 30:70 dry weight of the combination, preferably from 80:20 to 50:20 dry weight of the combination, most preferably from 70:30 to 60:40 dry weight of the combination. Suitably, the weight ratio of LNFP-I:LNDFH-I is 90:10, 85:15, 80:20, 75:25, 70:30, 65:35, 60:40, 55:45, 50:50, 45:55, 40:60, 35:65 or 30:70 dry weight of the combination.

In some embodiments of the combination, the weight ratio of total amount of LNFP:LNDFH-I is from 95:5 to 5:95 dry weight of the combination, preferably from 85:15 to 50:50 dry weight of the combination, most preferably from 80:20 to 60:40 dry weight of the combination. Suitably, the weight ratio of total LNFP:LNDFH-I is 95:5, 90:10, 85:15, 80:20, 75:25, 70:30, 65:35, 60:40, 55:45 or 50:50 dry weight of the combination.

In some embodiments of the combination or of the nutritional composition, LNFP-I is present in an amount ranging from 0.02 to 3.8 g/100 g or from 0.05 to 3.8 g/100 g or from 0.1 to 3.8 g/100 g or from 0.1 to 3 g/100 g or from 0.1 to 2.5 g/100 g or from 0.1 to 2 g/100 g or from 0.1 to 1.5 g/100 g or from 0.1 to 1 g/100 g or from 0.02 to 3 g/100 g or from 0.02 to 2.5 g/100 g or from 0.02 to 2 g/100 g or from 0.02 to 1.5 g/100 g or from 0.02 to 1 g/100 g or from or from 0.05 to 3 g/100 g or from 0.05 to 2.5 g/100 g or from 0.05 to 2 g/100 g or from 0.05 to 1.5 g/100 g or from 0.05 to 1 g/100 g or from 0.15 to 0.85 g/100 g or from 0.20 to 0.80 g/100 g or from 0.30 to 0.70 g/100 g or from 0.45 to 0.55 g/100 g dry weight of the combination or of the nutritional composition. Suitably, LNFP-I is present in an amount of 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.70, 0.80, 0.81, 0.82, 0.83, 0.84 or 0.85 g/100 g dry weight of the combination or of the nutritional composition.

In some embodiments of the combination or of the nutritional composition, the total amount of LNFP is present in an amount ranging from 0.02 to 3.8 g/100 g or from 0.05 to 3.8 g/100 g or from 0.1 to 3.8 g/100 g or from 0.1 to 3 g/100 g or from 0.1 to 2.5 g/100 g or from 0.1 to 2 g/100 g or from 0.1 to 1.5 g/100 g or from 0.1 to 1 g/100 g or from 0.02 to 3 g/100 g or from 0.02 to 2.5 g/100 g or from 0.02 to 2 g/100 g or from 0.02 to 1.5 g/100 g or from 0.02 to 1 g/100 g or from or from 0.05 to 3 g/100 g or from 0.05 to 2.5 g/100 g or from 0.05 to 2 g/100 g or from 0.05 to 1.5 g/100 g or from 0.05 to 1 g/100 g or from 0.15 to 0.85 g/100 g or from 0.20 to 0.80 g/100 g or from 0.30 to 0.70 g/100 g or from 0.45 to 0.55 g/100 g dry weight of the combination or of the nutritional composition. Suitably, the total amount of LNFP is present in an amount of 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.70, 0.80, 0.81, 0.82, 0.83, 0.84 or 0.85 g/100 g dry weight of the combination or of the nutritional composition. Suitably, the total amount of LNFP is present in an amount from less than 0.20, 0.25, 0.30, 0.35, 0.40, 0.45, 0.50, 0.55, 0.60, 0.65, 0.70, 0.75, 0.80 or 0.85 g/100 g dry weight of the combination or of the nutritional composition.

In some embodiments of the the combination or of the nutritional composition, LNDFHP-I is present in an amount ranging from 0.02 to 3.8 g/100 g or from 0.05 to 3.8 g/100 g or from 0.1 to 3.8 g/100 g or from 0.1 to 3 g/100 g or from 0.1 to 2.5 g/100 g or from 0.1 to 2 g/100 g or from 0.1 to 1.5 g/100 g or from 0.1 to 1 g/100 g or from 0.02 to 3 g/100 g or from 0.02 to 2.5 g/100 g or from 0.02 to 2 g/100 g or from 0.02 to 1.5 g/100 g or from 0.02 to 1 g/100 g or from or from 0.05 to 3 g/100 g or from 0.05 to 2.5 g/100 g or from 0.05 to 2 g/100 g or from 0.05 to 1.5 g/100 g or from 0.05 to 1 g/100 g or from 0.15 to 0.85 g/100 g or from 0.20 to 0.80 g/100 g or from 0.30 to 0.70 g/100 g or from 0.45 to 0.55 g/100 g dry weight of the combination or of the nutritional composition. Suitably, LNDFHP-I is present in an amount of 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.70, 0.80, 0.81, 0.82, 0.83, 0.84 or 0.85 g/100 g dry weight of the combination or of the nutritional composition.

In some embodiments of the combination or of the nutritional composition, LNDFHP-I is present in an amount ranging from 0.02 to 3.8 g/100 g or from 0.05 to 3.8 g/100 g or from 0.1 to 3.8 g/100 g or from 0.1 to 3 g/100 g or from 0.1 to 2.5 g/100 g or from 0.1 to 2 g/100 g or from 0.1 to 1.5 g/100 g or from 0.1 to 1 g/100 g or from 0.02 to 3 g/100 g or from 0.02 to 2.5 g/100 g or from 0.02 to 2 g/100 g or from 0.02 to 1.5 g/100 g or from 0.02 to 1 g/100 g or from or from 0.05 to 3 g/100 g or from 0.05 to 2.5 g/100 g or from 0.05 to 2 g/100 g or from 0.05 to 1.5 g/100 g or from 0.05 to 1 g/100 g or from 0.15 to 0.85 g/100 g or from 0.20 to 0.80 g/100 g or from 0.30 to 0.70 g/100 g or from 0.45 to 0.55 g/100 g dry weight of the combination or of the nutritional composition. Suitably, LNDFHP-I is present in an amount of 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.70, 0.80, 0.81, 0.82, 0.83, 0.84 or 0.85 g/100 g dry weight of the combination or of the nutritional composition.

In some embodiments of the combination or of the nutritional composition, the N-acetylated human milk oligosaccharide(s) is/are present in an amount ranging from 0.02 to 3.8 g/100 g or from 0.05 to 3.8 g/100 g or from 0.1 to 3.8 g/100 g or from 0.1 to 3 g/100 g or from 0.1 to 2.5 g/100 g or from 0.1 to 2 g/100 g or from 0.1 to 1.5 g/100 g or from 0.1 to 1 g/100 g or from 0.02 to 3 g/100 g or from 0.02 to 2.5 g/100 g or from 0.02 to 2 g/100 g or from 0.02 to 1.5 g/100 g or from 0.02 to 1 g/100 g or from or from 0.05 to 3 g/100 g or from 0.05 to 2.5 g/100 g or from 0.05 to 2 g/100 g or from 0.05 to 1.5 g/100 g or from 0.05 to 1 g/100 g or from 0.15 to 0.85 g/100 g or from 0.20 to 0.80 g/100 g or from 0.30 to 0.70 g/100 g or from 0.45 to 0.55 g/100 g dry weight of the combination or of the nutritional composition. Suitably, the N-acetylated human milk oligosaccharide(s) is/are present in an amount of 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.70, 0.80, 0.81, 0.82, 0.83, 0.84 or 0.85 g/100 g dry weight of the combination or of the nutritional composition.

In some embodiments of the combination or of the nutritional composition, the N-acetylated human milk oligosaccharide(s) comprises or is consisting of Lacto-N-tetraose (LNT) and/or LNnT, preferably consists of LNT.

Suitably, the nutritional composition may be provided in a serving size of 31 g total dry weight.

Suitably, the nutritional composition may be intended for administration twice daily.

Thus, the nutritional composition may be formulated to provide two servings per day.

According to the invention the nutritional composition comprises B. infantis as a probiotic.

In some embodiments, probiotic of the nutritional composition consists of B. infantis.

In some embodiments, the composition comprises the at least one probiotic in an amount of 1×10³ to 1.5×10¹² cfu/g of the composition (dry weight).

The nutritional composition according to the invention may contain from 10³ to 10¹² cfu of the at least one probiotic, more preferably between 10⁷ and 10¹² cfu such as between 10⁸ and 10¹⁰ cfu of the at least one probiotic per g of composition on a dry weight basis.

Suitably, the at least one probiotic is administered to the subject in an amount of at least about 10⁶ cfu/day, at least about 10⁷ cfu/day, or at least about 10⁸ cfu/day. Suitably, the at least one probiotic is administered to the subject in an amount of about 10¹² cfu/day or less, about 10¹¹ cfu/day or less, or about 10¹⁰ cfu/day or less.

In one embodiment the at least one probiotic is viable. In another embodiment the at least one probiotic is non-replicating or inactivated. There may be both viable probiotics and inactivated probiotics in some other embodiments. Probiotic components and metabolites can also be added.

Use to Enhance the Growth and/or Metabolic Activity of B. infantis

As described above, the combinations and compositions of the invention have surprisingly been found to enhance the growth of B. infantis.

In a further aspect, the present invention provides the use of the combination or nutritional composition of the invention for enhancing the growth and/or metabolic activity of B. infantis in the gastrointestinal tract of an infant, young child or child.

In a further aspect, the present invention provides a method of enhancing the growth and/or metabolic acitivity of B. infantis in the gastrointestinal tract of an infant, young child or child, the method comprising administering the combination or the nutritional composition of the invention to the infant, young child or child.

In a further aspect, the present invention provides the use of at least one probiotic for enhancing the growth and/or metabolic acitivity of B. infantis in the gastrointestinal tract of an infant, young child or child, wherein the at least one probiotic is as described herein elsewhere.

The combination or nutritional composition of the invention may prevent or treat an infection or a disease by enhancing the growth and/or metabolic activity of Bifidobacteria in the gastrointestinal tract of the subject.

The combination or the nutritional composition of the invention may prevent or treat an infection or a disease by increasing the levels of SCFA and/or other metabolites in the gastrointestinal tract and systemic levels of SCFA and/or other metabolites of the subject. The increased systemic levels of SCFA and/or other metabolites in the subject facilitates the exertion of the effects of the combination or nutritional composition of the invention beyond the gastrointestinal tract (e.g. in the lungs).

The combination or the nutritional composition of the invention may be used to treat or prevent disorders associated with decreased numbers and/or metabolic activity of Bifidobacteria in the gut (see e.g. Rivibre, A., et al., 2016. Frontiers in microbiology, 7, p. 979).

In one aspect, the invention provides the combination or the nutritional composition of the invention for use in treating and/or preventing a disorder associated with decreased numbers and/or metabolic activity of Bifidobacteria in the gut. In another aspect, the invention provides for use of the combination or the nutritional composition of the invention for the manufacture of a medicament for treating and/or preventing a disorder associated with decreased numbers and/or metabolic activity of Bifidobacteria in the gut. In another aspect, the invention provides a method of treating and/or preventing a disorder associated with decreased numbers and/or metabolic activity of Bifidobacteria in the gut in a subject, comprising administering the combination or the nutritional composition of the invention to the subject.

Suitably, the disorder associated with decreased numbers and/or metabolic activity of Bifidobacteria in the gut in a subject may be selected from: a gastrointestinal disease, obesity, an allergic disease, and regressive autism.

Accordingly, in a further aspect, the present invention provides the combination or the nutritional composition of the invention for use in enhancing the immune response to infection or vaccination in an infant, young child or child. In some embodiments, the antibody response to infection or vaccination is enhanced in the infant, young child or child.

In a further aspect, the present invention provides the use of the combination or the nutritional composition of the invention for the manufacture of a medicament for enhancing the immune response to infection or vaccination in an infant, young child or child in an infant, young child or child.

In a further aspect, the present invention provides a method of enhancing the immune response to infection or vaccination in an infant, young child or a child, the method comprising administering the combination or the nutritional composition of the invention to the infant, young child or child.

In some embodiments, the antibody response to infection or vaccination is enhanced in the infant, young child or child.

In some embodiments, the immune response to infection is enhanced.

In some embodiments, the immune response to vaccination is enhanced.

In a further aspect, the present invention provides the use of the combination or the nutritional composition of the invention for the manufacture of a medicament for preventing and/or reducing the risk of developing an infection in an infant, young child or child in an infant, young child or child.

In a further aspect, the invention provides a method of preventing and/or reducing the risk of developing an infection in an infant, young child or a child, the method comprising administering the combination or the nutritional composition of the invention to the infant, young child or child.

As used herein, the term “preventing and/or reducing the risk of developing an infection” includes preventing and/or reducing the risk of infection, delaying or preventing the onset of symptoms of infection and/or reducing the number or severity of symptoms of the infection.

The infection may be selected from respiratory tract infections (such as rhinovirus infections, respiratory syncytial virus infections, influenza virus infections), gastrointestinal tract infections (including Candida infections, healthcare-associated infections and healthcare-associated diarrhea, antibiotic associated infections and antibiotic associated diarrhea.

In some embodiments, the combination or the nutritional composition of the invention may be administered once daily. In other embodiments, the composition of the invention is administered in multiple servings, for example in two servings (also known as “unit doses”) per day. When the nutritional composition is provided in the form of unit doses (or “servings”) it is particularly useful to define the amount of oligosaccharides and probiotics in terms of the daily dose to be administered to the infant, or young child or child.

In some embodiments, a total amount of HMO ranging from 20 to 3800 mg/day (suitably, 50-800 mg/day or 100 to 600 mg/day) and B. infantis ranging from 10³ and 10¹² cfu/day are administered to the infant, young child or child. In one embodiment, a total amount of HMO ranging from 200 to 600 mg/day and B. infantis ranging from 10⁴ to 10⁹ cfu/day are administered to the infant, young child or child.

In some embodiments, an amount ranging from 50 to 950 mg/day of HMO and an amount ranging from 10³ to 10¹² cfu/day of B. infantis are administered to the infant, young child or child. In some embodiments, an amount ranging from 100 to 800 mg/day of HMO and an amount ranging from 10⁵ to 10⁹ cfu/day of B. infantis are administered to the infant, young child or child.

In some preferred embodiments, an amount ranging from 150 to 450 mg/day of HMO and an amount ranging from 10⁶ to 10⁸ cfu/day of B. infantis are administered to the infant, young child or child.

In some embodiments, about 50 mg/day (suitably, 100 mg/day, 150 mg/day, 200 mg/day, 250 mg/day, 300 mg/day, 350 mg/day, 400 mg/day, 450 mg/day, 500 mg/day, 550 mg/day, 600 mg/day, 650 mg/day, 700 mg/day, 750 mg/day, 800 mg/day, 850 mg/day, 900 mg/day or 950 mg/day) of HMO and about 10³ cfu/day (suitably, 10⁴ cfu/day, 10⁵ cfu/day, 10⁶ cfu/day, 10⁷ cfu/day, 10⁸ cfu/day, 10⁹ cfu/day, 10¹⁰ cfu/day, 10¹¹ cfu/day, 10¹² cfu/day) of B. infantis are administered to the infant, young child or child. In one embodiment, about 150 mg/day of HMO and about 10⁷ cfu/day of B. infantis are administered to the infant, young child or child. In one embodiment, about 300 mg/day of HMO and about 10⁹ cfu/day of B. infantis are administered to the infant, young child or child. In one embodiment, about 450 mg/day of HMO and about 10⁷ cfu/day of B. infantis are administered to the infant, young child or child. In one embodiment, about 50 mg/day of HMO and about 10⁷ cfu/day of B. infantis are administered to the infant, young child or child. In one embodiment, about 100 mg/day of HMO and about 10⁶ cfu/day of B. infantis are administered to the infant, young child or child. In one embodiment, about 150 mg/day of HMO and about 50 mg/day of B. infantis are administered to the infant, young child or child.

In some embodiments, the infant, young child or child is non-responsive to treatment B. infantis and LNFP-I.

The combination or the nutritional composition of the invention may be administered by any suitable method known to the skilled person. For example, the combination or the nutritional composition of the invention may be administered by oral and/or enteral administration. In some embodiments, the combination or the nutritional composition of the invention is orally administered.

Methods of Promoting and/or Maintaining Gastrointestinal Health

The use of probiotics, including Bifidobacteria strains, in preventive medicine to maintain a healthy intestinal function is well-documented (Tojo, R., et al., 2014. World journal of gastroenterology: WJG, 20(41), p. 15163). Further, the use of SCFA in preventive medicine to maintain a healthy intestinal function is also well-documented. The use of the combination or composition of the invention, which enhances the growth of Bifidobacteria and/or increases the levels of SCFA in the gastrointestinal tract of the subject would be expected to have the same effects.

In a further aspect, the present invention provides the combination or the nutritional composition of the invention for use in promoting and/or maintaining gastrointestinal health in an infant, young child or child.

In a further aspect, the present invention provides the use of the combination or the nutritional composition of the invention for the manufacture of a medicament for promoting and/or maintaining gastrointestinal health in an infant, young child or child.

In a further aspect, the present invention provides a method of promoting and/or maintaining gastrointestinal health in an infant, young child or a child, the method comprising administering the combination or the nutritional composition of the invention to the infant, young child or child.

In one aspect, the invention provides the use of the combination or the nutritional composition of the invention for promoting and/or maintaining gastrointestinal health. The combination or the nutritional composition of the invention may promote and/or maintain gastrointestinal health by enhancing the growth of Bifidobacteria, or by increasing the levels of SCFA, in the gastrointestinal tract of the subject.

Methods of Treating and/or Preventing a Gastrointestinal Disease

In some embodiments, promoting and/or maintaining gastrointestinal health includes treating and/or preventing a gastrointestinal disease.

In one aspect, the invention provides the combination or the nutritional composition of the invention for use in treating and/or preventing a gastrointestinal disease. In another aspect, the invention provides for use of the combination or the nutritional composition of the invention for the manufacture of a medicament for treating and/or preventing a gastrointestinal disease. In another aspect, the invention provides a method of treating and/or preventing a gastrointestinal disease in a subject, comprising administering the combination or the nutritional of the invention to the subject.

As used herein, a “gastrointestinal disease” (also known as “GI disease” or “GI illness”) may refer to diseases involving the gastrointestinal tract, which includes the oesophagus, stomach, small intestine, large intestine and rectum. In some embodiments, the gastrointestinal disease is a gastric disease or an intestinal disease.

In some embodiments, the gastrointestinal disease is a gastric disease. A “gastric disease” may refer to diseases affecting the stomach.

In some embodiments, the gastrointestinal disease is an intestinal disease. An “intestinal disease” may refer to diseases affecting the small intestine (including the duodenum, jejunum, and ileum) or large intestine (including the cecum, colon, and rectum).

In some embodiments, the gastrointestinal disease is selected from: healthcare-associateed diarrhea, antibiotic-associated diarrhea, Helicobacter pylori infection, an inflammatory bowel disease (IBD), irritable bowel syndrome (IBS), lactose intolerance, infectious diarrhea, and necrotizing enterocolitis.

### Antibiotic-Associated Diarrhea

A common complication of antibiotic use is the development of gastrointestinal disease. This complication ranges from mild diarrhea to pseudomembranous colitis. Antibiotic-associated diarrhea typically occurs in 5-35% of patients taking antibiotics and varies depending upon the specific type of antibiotic, the health of the host and exposure to pathogens. The pathogenesis of antibiotic-associated diarrhea may be mediated through the disruption of the normal microbiota resulting in pathogen overgrowth or metabolic imbalances (McFarland, L. V., 2008. Future Microbiology, 3(5), p. 563).

A probiotic mixture which contains several Bifidobacteria strains, among which are B. breve, B. infantis, and B. longum, displayed an ability to reduce the incidence of antibiotic-associated diarrhea (Selinger, C. P., et al., 2013. Journal of Hospital Infection, 84(2), pp. 159-165).

Helicobacter pylori Infection

Helicobacter pylori is a gram-negative microaerophilic bacterium that infects the epithelial lining of the stomach. Helicobacter pylori is the main cause of chronic gastritis and the principal etiological agent for gastric cancer and peptic ulcer disease. A recent global systematic review estimated that more than half the world's population is infected with Helicobacter pylori (Hooi, J. K., et al., 2017. Gastroenterology, 153(2), pp. 420-429).

A Helicobacter pylori eradication rate of 32.5% has been reported in adults after 10 days of administration of a probiotic mixture which contains several Bifidobacteria strains, among which are B. breve, B. infantis, and B. longum (Boltin, D., 2016. Best Practice & Research Clinical Gastroenterology, 30(1), pp. 99-109). Moreover, such a probiotic mixture has be shown to accelerate gastric ulcer healing (Dharmani, P., et al., 2013. PLoS One, 8(3), p. e58671).

### Inflammatory Bowel Disease

Inflammatory bowel disease (IBD) is a group of inflammatory conditions of the colon and small intestine. Exemplary IBDs include Crohn's disease (CD), ulcerative colitis (UC), and pouchitis. Is has been suggested that dysbiosis (that is, abnormal microbiota composition) and decreased complexity of the gut microbial ecosystem are common features in patients with IBD (see Manichanh, C., et al., 2012. Nature reviews Gastroenterology & hepatology, 9(10), pp. 599-608).

In IBD, a probiotic mixture which contains several Bifidobacteria strains, among which are B. breve, B. infantis, and B. longum, was able to reduce the UC symptoms in adults (Tursi, A., et al., 2010. The American journal of gastroenterology, 105(10), p. 2218) as well as the remission of the disease in children (Miele, E., et al., 2009. American Journal of Gastroenterology, 104(2), pp. 437-443).

In some embodiments, the IBD is Crohn's disease, ulcerative colitis, or pouchitis.

### Irritable Bowel Syndrome

Irritable bowel syndrome (IBS) is a functional bowel disorder characterised by chronic and recurrent abdominal pain and altered bowel habit (Chey, W. D., et al., 2015. Jama, 313(9), pp. 949-958). A growing body of evidence indicates dysbiosis as a hallmark of IBS (Rodino-Janeiro, B. K., et al., 2018. Advances in therapy, 35(3), pp. 289-310).

Administration of a probiotic mixture which contains several Bifidobacteria strains, among which are B. breve, B. infantis, and B. longum, for 6 weeks resulted in the reduction of IBS symptoms and the improvement of the quality of life in children (Guandalini, S., et al., 2010. Journal of pediatric gastroenterology and nutrition, 51(1), pp. 24-30).

### Other Gastrointestinal Diseases

Lactose intolerance is a common condition caused by a decreased ability to digest lactose. Administration of Bifidobacteria has been used to improve the symptoms of lactose intolerance (Hidalgo-Cantabrana, C., et al., 2017. Microbiology spectrum, 5(3), pp. 5-3).

Infectious diarrhea (also known as gastroenteritis) is inflammation of the gastrointestinal tract caused by an infection. Gastroenteritis is usually caused by viruses (e.g. rotavirus, norovirus, adenovirus, astrovirus, and coronavirus), however, bacteria (e.g. C. jeuni, E. coli, Salmonella, Shigella, C. difficile, and S. aureus), parasites (e.g. Giardia lamblia), and fungus can also cause gastroenteritis. There is evidence that that viable Bifidobacterium lactis has some protective effect against acute diarrhea in healthy children (Chouraqui, J. P., et al., 2004. Journal of pediatric gastroenterology and nutrition, 38(3), pp. 288-292).

Necrotizing enterocolitis (NEC) is an intestinal disease that affects premature infants. It has been shown that probiotic supplementation with Bifidobacteria can reduce both the incidence and severity of NEC in a premature neonatal population (Bin-Nun, A., et al., 2005. The Journal of pediatrics, 147(2), pp. 192-196).

In one aspect, the invention provides the combination or the nutritional composition of the invention for use in treating and/or preventing lactose intolerance, infectious diarrhea, or necrotizing enterocolitis. In another aspect, the invention provides for use of the combination or the nutritional composition of the invention for the manufacture of a medicament for treating and/or preventing lactose intolerance, infectious diarrhea, or necrotizing enterocolitis. In another aspect, the invention provides a method of treating and/or preventing lactose intolerance, infectious diarrhea, or necrotizing enterocolitis in a subject, comprising administering the combination or the nutritional composition of the invention to the subject.

### Subject

In some embodiments of the methods and uses of the invention, the combination or the nutritional composition is administered to an infant.

In some embodiments of the methods and uses of the invention, the combination or the nutritional composition is administered to a young child.

In some embodiments of the methods and uses of the invention, the combination or the nutritional composition is administered to a child.

In a further embodiment, the infant has an age ranging from 0 months to 12 months, for example 9 or 6 months.

In some embodiments, the young child has an age ranging from one year to three years, for example 2 years.

In some embodiments, the child has an age ranging from three years to nine years, for example from three years to seven years. In some preferred embodiments, the child has an age ranging from three years to five years, for example four years.

In preferred embodiments the subject is an infant, a young child or a child.

Some specific populations of infants, young children and children are particularly in need of combinations or nutritional compositions of the invention. Such infants, young children and children are for example preterm infants, low birth weight infant, and/or growth-retarded infants, young children or children. Indeed, such subjects are often experiencing adverse medical conditions and require significantly more frequent medical intervention than term infants and infants having experienced normal development.

The nutritional composition according to the invention is for use in infants, young children and/or children. The infants, young children and/or children may be born term or preterm. In a particular embodiment the nutritional composition of the invention is for use in infants, young children and/or children that were born preterm, having a low birth weight and/or born small for gestational age (SGA). In a particular embodiment the nutritional composition of the invention is for use in preterm infants, infants having a low birth weight and/or infants born small for gestational age (SGA).

The combination or the nutritional composition of the present invention may also be used in an infant, young child or child that was born by C-section or that was vaginally delivered.

In some embodiments the combination or the nutritional composition according to the invention can be for use before and/or during the weaning period. The combination or the nutritional composition can be administered (or given or fed) at an age and for a period that depends on the needs.

The combination or the nutritional composition can be for example given immediately after birth of the infants. The composition of the invention can also be given during the first week of life of the infant, or during the first 2 weeks of life, or during the first 3 weeks of life, or during the first month of life, or during the first 2 months of life, or during the first 3 months of life, or during the first 4 months of life, or during the first 6 months of life, or during the first 8 months of life, or during the first 10 months of life, or during the first year of life, or during the first two years of life or even more. In some particularly advantageous embodiments of the invention, the nutritional composition is given (or administered) to an infant within the first 4, 6 or 12 months of birth of said infant. In some other embodiments, the combination or the nutritional composition of the invention is given few days (e.g. 1, 2, 3, 5, 10, 15, 20 . . . ), or few weeks (e.g. 1, 2, 3, 4, 5, 6, 7, 8, 9, 10 . . . ), or few months (e.g. 1, 2, 3, 4, 5, 6, 7, 8, 9, 10 . . . ) after birth. This may be especially the case when the infant is premature, but not necessarily.

In one embodiment the nutritional composition of the invention is given to the infant or young child as a supplementary composition to the mother's milk. In some embodiments the infant or young child receives the mother's milk during at least the first 2 weeks, first 1, 2, 4, or 6 months. In one embodiment the nutritional composition of the invention is given to the infant or young child after such period of mother's nutrition, or is given together with such period of mother's milk nutrition. In another embodiment the composition is given to the infant or young child as the sole or primary nutritional composition during at least one period of time, e.g. after the 1^{st}, 2^{nd} or 4th month of life, during at least 1, 2, 4 or 6 months.

The subject may have or may be at risk of a low abundance and/or activity of Bifidobacteria in their gastrointestinal tract. The abundance of Bifidobacteria in the gastrointestinal tract may be determined by any method known to the skilled person (e.g. any method described in Tang, Q., et al., 2020. Frontiers in cellular and infection microbiology, 10, p. 151). The activity may be assessed by measuring typical Bifidobacteria metabolites like acetate for example using gas- and/or liquid chromatography coupled with mass spectrometry or another suitable detector.

A gastrointestinal tract sample may be obtained from or obtainable from fecal samples, endoscopy samples (e.g. biopsy samples, luminal brush samples, laser capture microdissection samples), aspirated intestinal fluid samples, surgery samples, or by in vivo models or intelligent capsule. Suitably, a gastrointestinal tract sample may be obtained from or obtainable from fecal samples. Fecal samples are naturally collected, non-invasive and can be sampled repeatedly.

The abundance of Bifidobacteria may be determined from the samples by any suitable method. For example, the abundance of Bifidobacteria may be obtained by or obtainable from the samples by sequencing methods (e.g. next-generation sequencing (NGS) methods), PCR-based methods, semi-quantitative detection methods, cycling temperature capillary electrophoresis, immunological-based methods, cell-based methods, or any combination thereof.

The subject may have or may be at risk of a disorder associated with decreased numbers of Bifidobacteria in the gut. Such disorders are described by Riviere, A., et al., 2016. Frontiers in microbiology, 7, p. 979, and may include gastrointestinal diseases, obesity, allergies, and regressive autism.

The subject may have or may be at risk of a gastrointestinal disease. In some embodiments, the subject may have or may be at risk of an antibiotic-associated diarrhea. In some embodiments, the subject may have or may be at risk of a Helicobacter pylori infection. In some embodiments, the subject may have or may be at risk of an IBD. In some embodiments, the subject may have or may be at risk of IBS. In some embodiments, the subject may have or may be at risk of lactose intolerance, infectious diarrhea, or necrotizing enterocolitis.

### Other Ingredients

The nutritional composition according to the present invention may also comprise non HMO oligosaccharide and/or fiberand/or precursor thereof that may be selected from the list comprising galacto-oligosaccharides (GOS), fructo-oligosaccharides (FOS), inulin, xylooligosaccharides (XOS), polydextrose and any combination thereof. They may be in an amount between 0 and 10% by weight of composition.

The nutritional composition of the present invention can further comprise at least one further probiotic (or probiotic strain), such as at least one further probiotic bacterial strain.

The probiotic microorganisms most commonly used are principally bacteria and/or yeasts of the following genera: Lactobacillus spp., Lacticaseibacillus spp, Limosilactobacillus spp, Streptococcus spp., Enterococcus spp., Bifidobacterium spp. and Saccharomyces spp.

In some particular embodiments, the probiotic is a probiotic bacterial strain. In some specific embodiments, it is particularly Bifidobacteria and/or Lactobacilli.

Suitable probiotic bacterial strains include Lactobacillus rhamnosus ATCC 53103 available from Valio Oy of Finland under the trademark LGG, Lactobacillus rhamnosus CGMCC 1.3724, Lactobacillus paracasei CNCM 1-2116, Lactobacillus johnsonii CNCM 1-1225, Streptococcus salivarius DSM 13084 sold by BLIS Technologies Limited of New Zealand under the designation K12, Bifidobacterium lactis CNCM 1-3446 sold inter alia by the Christian Hansen company of Denmark under the trademark Bb 12, B. longum CNCM 1-2618 (B. longum NCC2705), Bifidobacterium breve sold by Danisco under the trademark Bb-03, Bifidobacterium breve sold by Morinaga under the trade mark M-16V, Bifidobacterium infantis sold for example by Procter & Gamble Co. under the trademark Bifantis and Bifidobacterium breve sold by Institut Rosell (Lallemand) under the trademark R0070.

Suitable probiotic bacterial strains include Bifidobacterium animalis subsp. lactis CNCM 1-3446 deposited according to the Budapest Treaty on 7 Jun. 2005 at Collection Nationale Cultures De Microorganismes [French National Collection Of Microorganism Cultures](CNCM), Institut Pasteur, 25 Rue Du Docteur Roux, F-75724 Paris Cedex 15 (France), or BL818 or Bifidobacterium animalis subsp. lactis sold inter alia by the Christian Hansen company of Denmark under the trademark Bb 12, also known as DSM-15954, B. longum CNCM 1-2618 (B. longum NCC2705), Bifidobacterium breve sold by Danisco under the trademark Bb-03, Bifidobacterium breve sold by Morinaga underthe trade mark M-16V, Bifidobacterium breve sold by Morinaga under the trade mark B-3, Bifidobacterium breve sold by sold by Yakult under the trade mark BBG-01 and Bifidobacterium breve sold by Institut Rosell (Lallemand) under the trademark R0070.

In some preferred embodiments the Bifidobacterium longum subsp longum strain may be selected from Bifidobacterium longum subsp longum strain CNCM 1-2169, Bifidobacterium longum subsp longum strain CNCM 1-2171, Bifidobacterium longum subsp longum strain ATCC 15708, Bifidobacterium longum subsp longum strain DSM 20097, Bifidobacterium longum subsp longum strain NCIMB 8809, Bifidobacterium longum subsp longum strain CNCM 1-2618 (NCC 2705), Bifidobacterium longum subsp longum strain CNCM 1-2170, Bifidobacterium longum subsp longum strain ATCC 15707, or a combination thereof, in particular B. longum CNCM 1-2618 (NCC 2705).

Suitably, the Bifidobacterium longum subsp. longum has at least 99% (suitably, at least 99.1%, at least 99.2%, at least 99.3%, at least 99.4%, at least 99.5%, at least 99.6%, at least 99.7%, at least 99.8%, at least 99.9%) ANI to Bifidobacterium longum subsp. longum NCC 2705 (also known as Bifidobacterium longum subsp. longum CNCM 1-2618). Preferably, the Bifidobacterium longum subsp. longum has at least 99.9% ANI to Bifidobacterium longum subsp. longum NCC 2705.

B. longum NCC 2705 was deposited with the Institute Pasteur, 25 Rue Du Docteur Roux, F-75724 Paris Cedex 15 (France) according to the Budapest Treaty on 29 Jan. 2001 receiving the deposit no. CNCM 1-2618.

The nutritional composition according to the invention may contain from 10³ to 10¹² cfu of the at least one further probiotic strain, more preferably between 10⁷ and 10¹² cfu such as between 10⁸ and 10¹⁰ cfu of probiotic strain per g of composition on a dry weight basis.

In one embodiment the probiotics are viable. In another embodiment the probiotics are non-replicating or inactivated. There may be both viable probiotics and inactivated probiotics in some other embodiments. Probiotic components and metabolites can also be added.

The nutritional composition according to the invention can be for example an infant formula, a starter infant formula, a follow-on or follow-up formula, a growing-up milk, a baby food, an infant cereal composition, a fortifier such as a human milk fortifier, or a supplement. In some particular embodiments, the composition of the invention is an infant formula, a fortifier or a supplement that may be intended for the first 4 or 6 months of age. In a preferred embodiment the nutritional composition of the invention is an infant formula.

In some other embodiments the nutritional composition of the present invention is a fortifier. The fortifier can be a breast milk fortifier (e.g. a human milkfortifier) or a formula fortifier such as an infant formula fortifier or a follow-on/follow-up formula fortifier.

When the nutritional composition is a supplement, it can be provided in the form of unit doses. In such cases it is particularly useful to define the amount of oligosaccharides and probiotics in terms of daily dose to be administered to the infant, young child or child, such as described above.

The nutritional composition of the present invention can be in solid (e.g. powder), liquid or gelatinous form. In a specific embodiment the nutritional composition is a supplement, wherein the supplement is in powder form and provided in a sachet, preferably a sachet with 0.1 to 20 g per sachet, for example 1 to 10 g per sachet, or in the form of a syrup, preferably a syrup with a total solid concentration of 5 to 75 g/100 mL (5 to 75% (w/v)). When the supplement is in powder form, it may comprise a carrier. It is however preferred that the supplement is devoid of a carrier. When the supplement is in the form of a syrup, the components are preferably dissolved or suspended in water acidified with citrate.

The nutritional composition according to the invention generally contains a protein source. The protein can be in an amount of from 1.6 to 3 g per 100 kcal. In some embodiments, especially when the composition is intended for premature infants, the protein amount can be between 2.4 and 4 g/100 kcal or more than 3.6 g/100 kcal. In some other embodiments the protein amount can be below 2.0 g per 100 kcal, e.g. between 1.8 to 2 g/100 kcal, or in an amount below 1.8 g per 100 kcal.

Protein sources based on whey, casein and mixtures thereof may be used as well as protein sources based on soy. As far as whey proteins are concerned, the protein source may be based on acid whey or sweet whey or mixtures thereof and may include alpha-lactalbumin and beta-lactoglobulin in any desired proportions.

In some advantageous embodiments the protein source is whey predominant (i.e. more than 50% of proteins are coming from whey proteins, such as 60% or 70%).

The proteins may be intact or hydrolysed or a mixture of intact and hydrolysed proteins. By the term “intact” is meant that the main part of the proteins are intact, i.e. the molecular structure is not altered, for example at least 80% of the proteins are not altered, such as at least 85% of the proteins are not altered, preferably at least 90% of the proteins are not altered, even more preferably at least 95% of the proteins are not altered, such as at least 98% of the proteins are not altered. In a particular embodiment, 100% of the proteins are not altered.

The term “hydrolysed” means in the context of the present invention a protein which has been hydrolysed or broken down into its component amino acids. The proteins may be either fully or partially hydrolysed. It may be desirable to supply partially hydrolysed proteins (degree of hydrolysis between 2 and 20%), for example for infants or young children believed to be at risk of developing cow's milk allergy. If hydrolysed proteins are required, the hydrolysis process may be carried out as desired and as is known in the art. For example, whey protein hydrolysates may be prepared by enzymatically hydrolysing the whey fraction in one or more steps. If the whey fraction used as the starting material is substantially lactose free, it is found that the protein suffers much less lysine blockage during the hydrolysis process. This enables the extent of lysine blockage to be reduced from about 15% by weight of total lysine to less than about 10% by weight of lysine; for example about 7% by weight of lysine which greatly improves the nutritional quality of the protein source.

In an embodiment of the invention at least 70% of the proteins are hydrolysed, preferably at least 80% of the proteins are hydrolysed, such as at least 85% of the proteins are hydrolysed, even more preferably at least 90% of the proteins are hydrolysed, such as at least 95% of the proteins are hydrolysed, particularly at least 98% of the proteins are hydrolysed. In a particular embodiment, 100% of the proteins are hydrolysed.

In one particular embodiment the proteins of the nutritional composition are hydrolyzed, fully hydrolyzed or partially hydrolyzed. The degree of hydrolysis (DH) of the protein can be between 8 and 40, or between 20 and 60 or between 20 and 80 or more than 10, 20, 40, 60, 80 or 90.

The protein component can alternatively be replaced by a mixture or synthetic amino acids or peptides, for example for preterm or low birth weight infants.

In a particular embodiment the nutritional composition or the growing-up milk according to the invention is a hypoallergenic composition. In another particular embodiment the composition according to the invention is a hypoallergenic nutritional composition or growing-up milk.

The nutritional composition according to the present invention generally contains a carbohydrate source. This is particularly preferable in the case where the nutritional composition of the invention is an infant formula. In this case, any carbohydrate source conventionally found in infant formulae such as lactose, sucrose, saccharose, maltodextrin, starch and mixtures thereof may be used although one of the preferred sources of carbohydrates is lactose.

The nutritional composition according to the present invention generally contains a source of lipids. This is particularly relevant if the nutritional composition of the invention is an infant formula. In this case, the lipid source may be any lipid or fat which is suitable for use in infant formulae. Some suitable fat sources include palm oil, structured triglyceride oil, high oleic sunflower oil and high oleic safflower oil, medium-chain-triglyceride oil. The essential fatty acids linoleic and α-linolenic acid may also be added, as well small amounts of oils containing high quantities of preformed arachidonic acid and docosahexaenoic acid (DHA) such as fish oils or microbial oils. The fat source may have a ratio of n-6 to n-3 fatty acids of about 5:1 to about 15:1; for example about 8:1 to about 10:1.

The nutritional composition of the invention may also contain all vitamins and minerals understood to be essential in the daily diet and in nutritionally significant amounts. Minimum requirements have been established for certain vitamins and minerals. Examples of minerals, vitamins and other nutrients optionally present in the composition of the invention include vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chlorine, potassium, sodium, selenium, chromium, molybdenum, taurine, and L-carnitine. Minerals are usually added in salt form. The presence and amounts of specific minerals and other vitamins will vary depending on the intended population.

If necessary, the nutritional composition of the invention may contain emulsifiers and stabilisers such as soy, lecithin, citric acid esters of mono- and diglycerides, and the like.

The nutritional composition of the invention may also contain other substances which may have a beneficial effect such as lactoferrin, nucleotides, nucleosides, and the like.

The nutritional composition of the invention may also contain carotenoid(s) such as lutein (free lutein, lutein esters, lutein salts). In some embodiments, the weight ratio of lutein (μg) to docosahexaenoic acid (mg) is from 1.5:1 to 10:1, preferably from 2:1 to 5:1. In some embodiments of the invention, the nutritional composition of the invention does not comprise any carotenoid.

The nutritional composition according to the invention may be prepared in any suitable manner. A composition will now be described by way of example.

For example, a formula such as an infant formula may be prepared by blending together the protein source, the carbohydrate source and the fat source in appropriate proportions. If used, the emulsifiers may be included at this point. The vitamins and minerals may be added at this point but they are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture. The temperature of the water is conveniently in the range between about 50° C. and about 80° C. to aid dispersal of the ingredients. Commercially available liquefiers may be used to form the liquid mixture. The fucosylated oligosaccharide(s) and the optional N-acetylated oligosaccharide(s) may be added at this stage, especially if the final product is to have a liquid form. If the final product is to be a powder, they may likewise be added at this stage if desired.

The liquid mixture is then homogenised, for example in two stages.

The liquid mixture may then be thermally treated to reduce bacterial loads, by rapidly heating the liquid mixture to a temperature in the range between about 80° C. and about 150° C. for a duration between about 5 seconds and about 5 minutes, for example. This may be carried out by means of steam injection, an autoclave or a heat exchanger, for example a plate heat exchanger.

Then, the liquid mixture may be cooled to between about 60° C. and about 85° C. for example by flash cooling. The liquid mixture may then be again homogenised, for example in two stages between about 10 MPa and about 30 MPa in the first stage and between about 2 MPa and about 10 MPa in the second stage. The homogenised mixture may then be further cooled to add any heat sensitive components, such as vitamins and minerals. The pH and solids content of the homogenised mixture are conveniently adjusted at this point.

If the final product is to be a powder, the homogenised mixture is transferred to a suitable drying apparatus such as a spray dryer or freeze dryer and converted to powder. The powder should have a moisture content of less than about 5% by weight. The oligosaccharide(s) may also or alternatively be added at this stage by dry-mixing or by blending them in a syrup form of crystals, along with the probiotic strain(s), and the mixture is spray-dried or freeze-dried.

If a liquid composition is preferred, the homogenised mixture may be sterilised then aseptically filled into suitable containers or may be first filled into the containers and then retorted.

In another embodiment, the composition of the invention may be a supplement. The supplement may be in the form of tablets, capsules, pastilles or a liquid for example. The supplement may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents and gel forming agents. The supplement may also contain conventional pharmaceutical additives and adjuvants, excipients and diluents, including, but not limited to, water, gelatine of any origin, vegetable gums, lignin-sulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like.

Further, the supplement may contain an organic or inorganic carrier material suitable for oral or parenteral administration as well as vitamins, minerals trace elements and other micronutrients in accordance with the recommendations of Government bodies such as the USRDA.

In one embodiment, the nutritional composition is comprising
From 0.02% to 5%, preferably from 0.1% to 1.8% of at least one human milk oligosaccharide based on the total amount of the nutritional composition on a dry weight basis,
from 10% to 40%, preferably from 20% to 30% of at least one source of lipids, such as include palm oil, structured triglyceride oil, high oleic sunflower oil and high oleic safflower oil, medium-chain-triglyceride oil. The essential fatty acids linoleic and α-linolenic acid may also be added, as well small amounts of oils containing high quantities of preformed arachidonic acid and docosahexaenoic acid such as fish oils or microbial oils,
from 10³ to 10¹², preferably from 10⁵ to 10¹⁰ cfu of a Bifidobacterium longum subspecies infantis (B. infantis), preferably LMG 11588 and/or ATCC 15697;
from 0.1% to 1.5%, preferably from 0.5% to 1% of at least one vitamin and/or mineral such as vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chlorine, potassium, sodium, selenium, chromium, molybdenum, taurine, and L-carnitine,
from 2% to 20%, preferably from 5% to 15% of at least one protein source,
from 30% to 80%, preferably from 45% to 65% of a carbohydrate source, preferably lactose, sucrose, saccharose, maltodextrin, starch and mixtures thereof, more preferably lactose,
optionally at least one of emulsifiers and stabilisers such as soy, lecithin, citric acid esters of mono- and diglycerides, substances which may have a beneficial effect such as lactoferrin, nucleotides, nucleosides, and carotenoid;
wherein the % by weight of the at least one human milk oligosaccharide in the nutritional composition on a dry weight basis is
(a) 0.015% to 3.8%, preferably from 0.08% to 1.2% of lacto-N-fucopentaose I (LNFP-I),
(b) from 0% to 3.8%, more preferably from 0.08% to 1.5% of at least one fucohexaose, preferably Lacto-N-fucohexaose-I (LNDFH-I),
(c) 0% to 3%, preferably from 0.08% to 1.2% of lacto-N-fucopentaose II (LNFP-II),
(d) 0% to 3.8%, preferably from 0.04% to 1.2% of lacto-N-fucopentaose Ill (LNFP-III),
(e) Optionally 0% to 1.5%, preferably from 0.04% to 2.3% of at least one N-acetylated human milk oligosaccharide, preferably Lacto-N-tetraose (LNT), Lacto-N-neotetraose (LNnT) or a mixture thereof,
(f) Optionally 0% to 1.9%, preferably from 0.4% to 1.2%, more preferably 0.6% to 1.2 of at least one further human milk oligossacharide other than (a) to (e).

### EXAMPLES

### Materials and Methods

### Example 1

### Isolation of an HMO blend enriched in Lacto-N-fucopentaoses (LNFP) and Lacto-N-fucohexaose-I (LNDFH-1)

The HMO blend was prepared as follows: Human milk was skimmed after centrifugation. Ethanol was added to skimmed milk to a final concentration of approximately 66% (v/v). After overnight incubation at −20° C. the formed precipitates were removed by centrifugation and decantation through a paper filter. The solution was freeze dried and taken up again in MilliQ water. The solution was loaded and separated on a preparative Bio-Gel P-2 Gel (Bio-Rad, Hercules, California, USA) size exclusion column. Fractions were collected and analysed using a HPAEC (high performance anion exchange chromatography) chromatography system equipped with a CarboPac PA1 column (Thermo Fisher Scientific, Dionex, California, USA) and a pulsed amperometry detector.

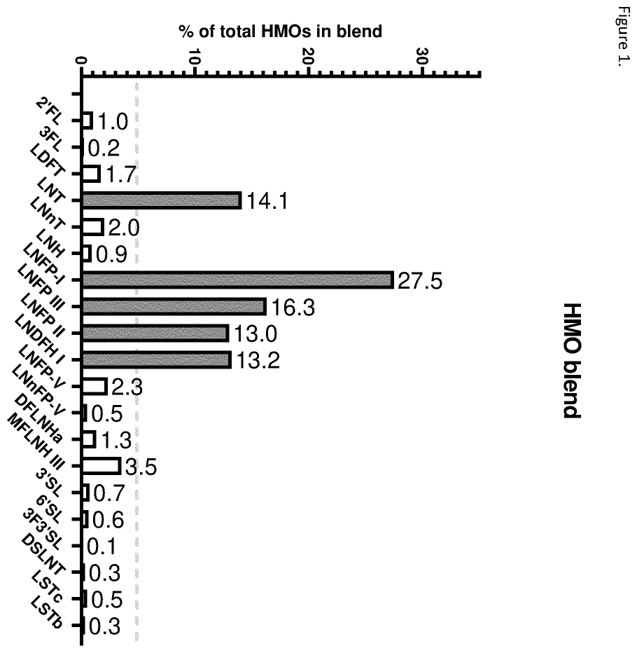

Fractions containing the LNFPs, LNDFH-I and LNT were pooled and freeze dried. FIG. 1 shows the relative abundance of the individual HMOs in the obtained blend, dubbed HMO blend.

The composition of the HMO blend of obtained is shown in FIG. 1.

### Example 2

Neutralisation of Enteropathogenic E. coli and Salmonella with a Combination of the an HMO Blend with B. infantis According to the Invention.

A combination of the HMO blend prepared in example 1 with Bifidobacterium longum subspecies infantis (B. infantis, ATCC 15697) was tested as follows. The combination with the HMO blend at 1% (w/v) was grown with a Bifidobacterium longum subspecies infantis (B. infantis, ATCC 15697) starting from a single colony over night at 37° C. in anaerobic conditions with commercially available glucose-free MRS (DeMan, Rogosa and Sharpe) broth. In parallel other glycans of the comparative examples were also incubated at 1% (w/v). Each overnight culture was diluted to have a starting OD600 of 0.1 in DMEM (Dulbeccos modified Eagle Medium) containing as a carbon source 0.1% glucose. This medium was used without any further carbohydrate supplement or with additional 1% (w/v) HMO blend, or 1% (w/v) of other glycans. Conditioning of DMEM media was thus done at 37° C. anaerobic during overnight incubation. Conditioned media were then centrifuged and supernatants filtered through a 0.22 micrometer filter to remove B. infantis. The culture media were centrifuged and filtered to obtain a spent culture medium prepared or other glycans as comparative examples.

Spent culture media were incubated at different dilutions with Salmonella typhymurium and Enteropathogenic E. coli. For Salmonella growth over time was monitored. For Salmonella and EPEC cells were enumerated after plating.

### COMPARATIVE EXAMPLES

Neutralisation of Enteropathogenic E. coli and Salmonella with Glycans not According to the Invention with B. infantis.

The procedure of example 2 was repeated 6 times with the exception that the HMO blend prepared in example 1 was replaced each time with
Glucose (Glc),
Lactose (Lac),
2′Fucosyllactose (2′FL),
Lacto-N-tetraose (LNT),
Lacto-N-neotetraose (LNnT),
6′Sialyllactose (6′SL).

### Results.

The HMO blend enriched in LNFP-1 according to the invention showed strongest neutralization activity of both Salmonella typhymurium and enteropathogenic E. coli. compared to Glucose (Glc), Lactose (Lac), 2′Fucosyllactose (2′FL), Lacto-N-tetraose (LNT), Lacto-N-neotetraose (LNnT) or 6′Sialyllactose (6′SL). Especially neither LNT nor 2′FL had the same effect on Salmonella typhymurium growth as the HMO blend according to the invention, whereas LNT and 2′FL are recognized as suitable B. infantis substrates, hence, without being bound by any theory, it can be supposed that another mechanism of action is triggered with HMO blend according to the invention.

The best inhibition of pathogen growth is observed with the HMO blend enriched in LNFP-I according to the invention (example 1), whereas the combination of B. infantis with LNT alone results in a lower inhibition, hence, it is highly plausible that the inhibition (neutralization) effects observed with the HMO blend enriched in LNFP-I according to the invention are due to the HMOs LNFP-I, -II and -III and LNDFH-I, more particularly the most abundant one LNFP-I, and not due to any N-acetylated human milk oligosaccha ride, especially LNT.

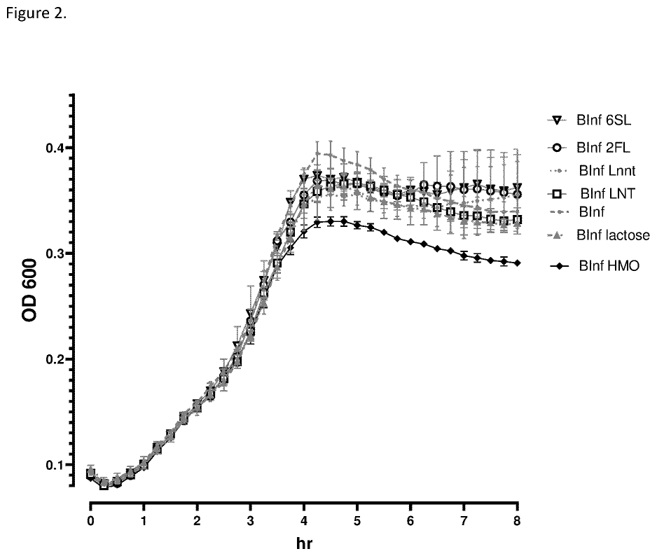

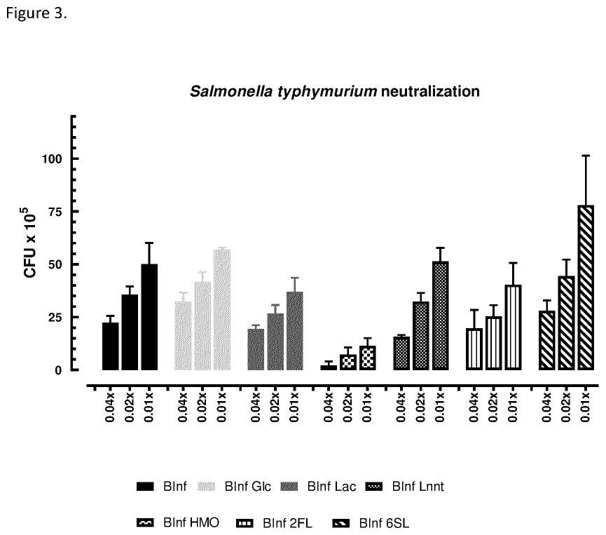

The neutralization of Salmonella typhymurium is illustrated in FIGS. 2 and 3.

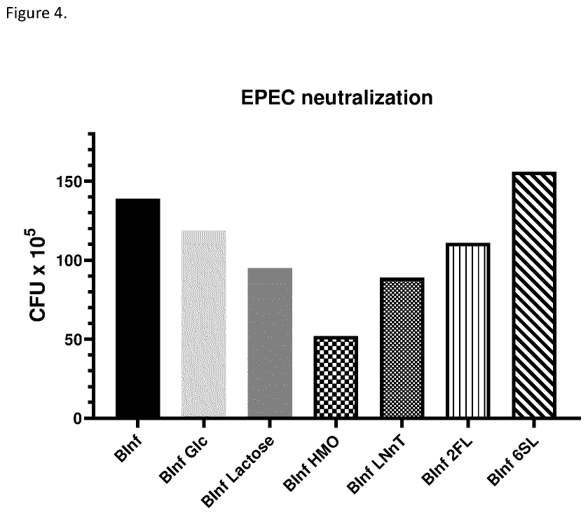

The neutralization of Enteropathogenic E. coli is illustrated in FIG. 4.

Growth was assessed by monitoring the optical density at 600 nm using a photometer.

### Example 3

An example of the composition of an infant formula according to the present invention is given in the below table 1. This composition is given by way of illustration only.

TABLE 1
Composition of the infant formula of Example 1

| Nutrients | per 100 kcal | per litre |
|---|---|---|
| Energy (kcal) | 100 | 670 |
| Protein (g) | 1.83 | 12.3 |
| Fat (g) | 5.3 | 35.7 |
| Linoleic acid (g) | 0.79 | 5.3 |
| α-Linolenic acid (mg) | 101 | 675 |
| Lactose (g) | 11.2 | 74.7 |
| Minerals (g) | 0.37 | 2.5 |
| Na (mg) | 23 | 150 |
| K (mg) | 89 | 590 |
| Cl (mg) | 64 | 430 |
| Ca (mg) | 62 | 410 |
| P (mg) | 31 | 210 |
| Mg (mg) | 7 | 50 |
| Mn (μg) | 8 | 50 |
| Se (μg) | 2 | 13 |
| Vitamin A (μg RE) | 105 | 700 |
| Vitamin D (μg) | 1.5 | 10 |
| Vitamin E (mg TE) | 0.8 | 5.4 |
| Vitamin K1 (μg) | 8 | 54 |
| Vitamin C (mg) | 10 | 67 |
| Vitamin B1 (mg) | 0.07 | 0.47 |
| Vitamin B2 (mg) | 0.15 | 1.0 |
| Niacin (mg) | 1 | 6.7 |
| Vitamin B6 (mg) | 0.075 | 0.50 |
| Folic acid (μg) | 9 | 60 |
| Pantothenic acid (mg) | 0.45 | 3 |
| Vitamin B12 (μg) | 0.3 | 2 |
| Biotin (μg) | 2.2 | 15 |
| Choline (mg) | 10 | 67 |
| Fe (mg) | 1.2 | 8 |
| I (μg) | 15 | 100 |
| Cu (mg) | 0.06 | 0.4 |
| Zn (mg) | 0.75 | 5 |
| HMO(s) (g) from combination of | 0.34 | 2.3 |
| example 1 | | |
| Other HMO(s) (g) | 0 to 1.32 | 0 to 6.7 |
| B. infantis from combination | 1.4 × 10⁷ cfu | 10⁸ cfu |

### Example 4

### In Vitro Growth Assays

B. infantis LMG11588 was grown in anaerobic conditions in minimal medium MRS API with 0.25% Glucose overnight. After 16 h growth, this overnight culture was used to inoculate B. infantis LMG11588 at OD 0.05 in MRS API medium supplemented with 0.25% of below listed carbon sources in a total volume of 1200 μL (microliter).

Following conditions were tested:
Glucose (positive control)
No substrate, in which no sugar was added (negative control)
LNFP-I
LNT

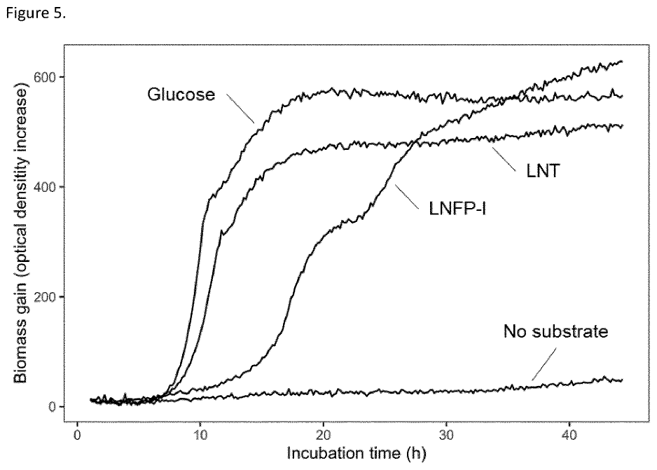

Growth was conducted at 37° C., under anaerobic conditions (CO2), for 48 hours, without pH control. Initial conditions were set at pH 5.7. Biomass gain was continuously monitored over time to evaluate if the selected carbon sources could support the growth of B. infantis LMG11588. Experiments were conducted in triplicate, with the average being depicted in FIG. 5.

The biomass gain was continuously monitored as a measure of if and how the different carbon sources could support the growth of B. infantis LMG11588 over the course of 48 hours.

When no carbon source was added (no substrate), no growth, measured as biomass gain, was observed, whilst the addition of the simple sugarglucose or the less complex HMO LNT resulted in exponential growth that started to stagnate after 10 h. B. infantis LMG11588 can utilize LNFP-I to grow, and after an adaptation period, a diauxic growth is observed, showcasing that B. infantis is able to utilize LNFP-I as a sole carbon source to ensure increase of biomass.

It should be understood that various changes and modifications to the presently preferred embodiments described herein will be apparent to those skilled in the art. Such changes and modifications can be made without departing from the spirit and scope of the present invention and without diminishing its attendant advantages. It is therefore intended that such changes and modifications be covered by the appended claims, as long as they remain within the scope of the independent claims.

1: A method of treating, preventing and/or inhibiting growth of at least one enteric pathogen in a subject, the method comprising administering a composition to the subject,
the composition comprising at least one human milk oligosaccharide (HMO), the at least one HMO comprising at least one fucosylated HMO, the at least one fucosylated HMO comprising at least one lacto-N-fucopentaose, the at least one lacto-N-fucopentaose comprising lacto-N-fucopentaose-I (LNFP-I),
wherein the LNFP-I is the most abundant HMO in the composition,
wherein the composition further comprises at least one Bifidobacterium longum subspecies.

2: The method according to claim 1, wherein the lacto-N-fucopentaose I (LNFP-I) represents at least 5% by weight of a total amount of the at least one human milk oligosaccharidein the composition on a dry weight basis.

3: The method according to claim 1, wherein the at least one lacto-N-fucopentaose further comprises at least one additional lacto-N-fucopentaose other than LNFP-I.

4: The method according to claim 1, wherein the at least one fucosylated HMO further comprises at least one fucohexaose.

5: The method according to claim 1, wherein a total amount of the at least one lacto-N-fucopentaose represents at least 5% by weight of a total amount of the at least one human milk oligosaccharidein the composition on a dry weight basis.

6: The method according to claim 4, wherein the at least one fucohexaose is at least 5% by weight of a total amount of the at least one human milk oligosaccharide in the composition on a dry weight basis.

7: The method according to claim 4, wherein the at least one fucohexaose comprises of Lacto-N-fucohexaose-I (LNDFH-I).

8: The method according to claim 1, wherein the at least one enteric pathogen is Salmonella typhymurium.

9: The method according to claim 1, wherein the at least one enteric pathogen is enteropathogenic E. coli (EPEC).

10: The method according to claim 1, wherein the at least one Bifidobacterium longum subspecies infantis is LMG 11588, ATCC 15697 (NCC 341) or a mixture thereof.

11: The method according to claim 1, the at least one HMO further comprises at least one N-acetylated human milk oligosaccharide.

12: The method according to claim 1, wherein the composition is a nutritional composition.

13: The method according to claim 12, wherein the nutritional composition is an infant formula, a starter infant formula, a follow-on or follow-up infant formula, a growing-up milk, a baby food, an infant cereal composition, a milk fortifier or a supplement.

14. (canceled)

15: A method for promoting immunity in a subject in need thereof, the method comprising administering a composition to the subject,
the composition comprising at least one human milk oligosaccharide (HMO), the at least one HMO comprising at least one fucosylated HMO, the at least one fucosylated HMO comprising at least one lacto-N-fucopentaose, the at least one lacto-N-fucopentaose comprising lacto-N-fucopentaose-I (LNFP-I),
wherein the LNFP-I is the most abundant HMO in the composition,
wherein the composition further comprises at least one Bifidobacterium longum subspecies infantis.

16: The method according to claim 15, wherein the composition is a nutritional composition, wherein the nutritional composition comprises
from 0.02% to 5% of the at least one human milk oligosaccharide based on the total amount of the nutritional composition on a dry weight basis,
from 10% to 40% of at least one source of lipids,
from 10³ to 10¹² cfu of the Bifidobacterium longum subspecies infantis (B. infantis),
from 0.1% to 1.5% of at least one vitamin and/or mineral,
from 2% to 20% of at least one protein source, amd
from 30% to 80% of a carbohydrate source,
wherein the % by weight of the at least one human milk oligosaccharide in the nutritional composition on a dry weight basis is
(a) 0.015% to 3.8% of the lacto-N-fucopentaose I (LNFP-I),
(b) from 0% to 3.8% of at least one fucohexaose,
(c) 0% to 3% of lacto-N-fucopentaose II (LNFP-II), and
0% to 3.8% of lacto-N-fucopentaose III (LNFP-III).

17: The method according to claim 1, wherein the subject is an infant, a young child or a child.

18: The method according to claim 1, wherein the lacto-N-fucopentaose I (LNFP-I) is 25% to 95% by weight of a total amount of the at least one human milk oligosaccharides in the composition.

19: The method according to claim 3, wherein the at least one additional lacto-N-fucopentaose other than LNFP-I comprises both lacto-N-fucopentaose II (LNFP-II) and lacto-N-fucopentaose III (LNFP-III).

20: The method according to claim 1, wherein a total amount of the at least one lacto-N-fucopentaose is 30% to 95% by weight of a total amount of the at least one human milk oligosaccharide in the composition on a dry weight basis.
